(19) United States
(12) Patent Application Publication
(10) Pub. No.: US 2022/0249687 A1
(43) Pub. Date: Aug. 11, 2022

(54) BIS(2-HALOACETAMIDO)-COMPOUNDS FOR USE AS LINKING AGENTS AND RESULTANT PRODUCTS WHICH COMPRISE ANTIBODIES, HALF-ANTIBODIES AND ANTIBODY FRAGMENTS

(71) Applicant: THE UNIVERSITY OF BATH, Bath and North East Somerset (GB)

(72) Inventors: Andrew Watts, Bath Somerset (GB); Bayan Alkhawaja, Bath Somerset (GB)

(21) Appl. No.: 17/622,662

(22) PCT Filed: Jun. 25, 2020
(86) PCT No.: PCT/EP2020/067922

§ 371 Date: Dec. 23, 2021

(30) Foreign Application Priority Data
Jun. 25, 2019 (GB) 1909103.2

(51) Int. Cl. A61K 47/68; C07C 233/54; C07D 207/46; C07C 233/43; C07K 16/32; C07K 16/28

(52) U.S. Cl. CPC A61K 47/6889; C07C 233/54; C07D 207/46; C07K 2317/55; C07K 16/32; C07K 16/2887; C07K 2317/53; C07C 233/43

(57) ABSTRACT

Bis(2-haloacetamido)-compounds for use as linkers to chemically cross-linking multiple thiol groups, and particularly, although not exclusively, the thiol groups of cysteine amino acids in peptide chains are described, along with their use as linking agents and resultant products which comprise antibodies, half-antibodies and antibody fragments having thiol groups bonded to said linkers (e.g. antibody-protein conjugates and antibody-drug conjugates), and methods of making said conjugates and products. (Formula I)

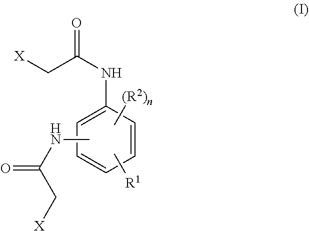

### FIELD OF THE INVENTION

The present invention relates to the use of bis(2-haloacetamido)-derivatives as compounds for chemically cross-linking multiple thiol groups, and particularly, although not exclusively, to the thiol groups of cysteine amino acids in peptide chains.

The invention therefore provides aryl bis(2-haloacetamido)-compounds for use as linking compounds; antibody, half-antibody and antibody fragment conjugates having thiol groups bonded to said linkers, products which comprise antibodies, half-antibodies and antibody fragments having thiol groups bonded to said linkers (e.g. antibody-protein conjugates and antibody-drug conjugates), and to methods of making said conjugates and products.

### BACKGROUND

Antibody drug conjugates (ADCs) are one of the cutting-edge antibody-based therapeutic approaches. As such, they have attracted substantial attention over the last two decades. ADCs comprise a monoclonal antibody (mAb) which is employed as a vehicle to direct a cytotoxic drug to a specific malignant cell or tissue. The construction of ADCs requires specific linker characteristics in order to attain plasma-stable antibody drug conjugates.

The construction of the first-generation of ADCs utilised the inherent nucleophilic functionality of the amino acid residues within the antibody, mainly cysteine and lysine amino acids. In order to generate cysteine-based conjugates, a pre-reduction step of inter-chain disulfide bonds is required. The IgGi subclass of immunoglobulin G antibodies (IgGs) is the most abundant one in human serum and most common type employed in mAb-based therapeutics. IgGi has a total of four inter-chain disulfide bonds, two inter-chain disulfide bonds are between heavy/light chains (HC/LC) and two inter-chain between heavy/heavy chains (HC/HC) in the hinge region.

A great number of studies have shown the importance of the 4 inter-chain disulfide bonds, not only to maintain the structure of mAbs and optimal antigen binding, but also to reserve the effector function of mAbs. A reduction in Complement Dependent Cytotoxicity (CDC) and Antibody-Dependent Cell-Mediated Cytotoxicity (ADCC) were observed for a chimeric mouse/human IgG when a mutated IgG lacking inter heavy chain disulfide bonds was tested.

It has been found that even partial reduction of the four inter-chain disulfide bonds of IgG results in a dramatic reduction in their complement fixation function and CDC, while the usefulness of half-antibodies is increasingly recognised.

Given that disulfide bonds are essential to maintain the proper folding, structure and more importantly, the function of mAbs, it is essential to maintain the post-reduction structure of recombinant antibodies prior to the construction of mAb conjugates. In attempts to avoid the complex and expensive genetic re-engineering of mAbs to maintain the post-reduction structure of mAbs and retain a defined conjugation approach, Abzena has pioneered a novel approach (ThioBridge® technology) of producing homogenous and stable ADCs based on rebridging of the reduced inter-chain disulfide bonds.

ThioBridge® technology is a bis-sulfone-based reagent which re-anneals the reduced inter-chain disulfide bonds of mAbs forming a 3-carbon bridge, and by doing so, ThioBridge® maintains the post-reduced disulfide structure of the antibody. The accessible reduced disulfide bonds are cross-linked through sequential addition-elimination mechanism, first starting by Michael addition of a thiolate group of the reduced disulfide bond, followed by elimination of p-toluene sulfinic acid generating the second Michael acceptor (α-β unsaturated carbonyl). The vicinal thiolate group then reacts through a second Michael addition to form a 3-carbon linkage between the two cysteine thiols.

Given that the primary aim of using bis-sulfone linkers is to attain better plasma stability of the ADCs, the increased plasma stability was appraised by comparing fluorescently-labeled Trastuzumab using a maleimide linkage, with fluorescently-labeled bis-alkylated Trastuzumab. The latter displayed significant serum stability over 5 days (compared with the maleimide linked antibody) in the presence of high concentrations of albumin.

Moreover, it has been confirmed that using a bis-sulfone reagent bearing monomethyl auristatin E (MMAE) in the construction of ADCs of Trastuzumab, fully retained binding activity of Trastuzumab to the HER2 receptor, and displayed potent antiproliferative activity in HER2 positive cell lines.

Bis-reactive maleimide linkers have been developed as disulfide rebridging agents through 2-carbon bridge to attain bis-thiosuccinamide linkage. The fast reaction kinetics of bis-reactive maleimide derivatives offers higher protein structure integrity and broader reaction pH range (6-8) compared to bis-sulfone derivatives.

Bis-sulfone reagents and dibromomaeimide derivatives are deemed as the earliest and most established rebridging reagents for reduced disulfides. Nonetheless, each has associated drawbacks. These include poor water solubility of the reagents and lack of selectivity between reduced disulfides bonds and unpaired cysteine residues under reducing conditions. Bridging can occur across both heavy-light and heavy-heavy chains, leading to a mixture of products.

There is therefore interest in finding further linkers for replacing the sulfide bridges in antibodies.

Other example scaffolds include pyridazinedione-based compounds, such as dibromopyridazinedione, which cross-links disulfide bonds through a 2-carbon bridges. Di-TCEP-substituted dithiophenolpyridazinedione has also been used, and can function as both a reducing agent and stable cross-linker at the same time. Other approaches have introduced clickable chemistry onto pyridazinedione-based linkers to permit the inclusion of additional functionality.

EP3335734 relates to the use of drug moieties attached to linkers which can be used to form anti-drug conjugates through displacement of leaving groups to form thioethers. For example, auristatin E (MMAE) and auristatin F were attached to linkers via amide coupling. Herceptin-linker-drug units are disclosed.

Rebridging strategies are considered elegant approaches that enable the introduction of reactive functional groups to the reduced mAbs while maintaining the well-defined post-reduction structure of them. Despite the recent advances and considerable research efforts in this area, the aforementioned rebridging approaches either through forming 2- and 3-carbon bridges still have practical limitations that needed to be further addressed. These include poor yield and mixtures of products being produced.

The present invention has been devised in light of the above considerations.

### SUMMARY OF THE INVENTION

The present invention provides aryl bis(2-haloacetamido)-derivatives as next-generation rebridging agents. These bis-haloacetamide derivatives have utility in conventional rebridging as described above, but may be especially useful in the hetero rebridging (attachment of two or more different rebridging linkers) of full mAbs and in the production of half-antibody conjugates.

With all major emphasis being on fully cross-linking the heavy-light and heavy-heavy inter-chain disulfide bonds to attain a fully cross-linked antibody (150 KDa), the previously described (see background) re-bridging methods have overlooked the potential advantages of construction of a half-antibody (75 KDa) as an intended product. For example, half-antibodies may have utility in endosomal recycling and tumour penetration. In addition, to the best of the inventors' knowledge, the previous rebridging approaches have shown no significant selectivity between heavy-heavy and heavy-light disulfide bonds. Therefore, site-selective protein labelling to attain a hetero-bifunctional mAb was a previously unresolved challenge.

It is an aim of the present invention to address these problems.

Linker Compounds

In a first aspect, the invention provides linker compounds bearing a bis-(2-haloacetamide) motif. The linkers are suitable for rebridging (linking) sulfide bridges in antibody structures and/or linking heavy chains intra, rather than inter, to form half-antibodies. These linked antibody structures are termed “conjugates”.

Accordingly, in a first aspect the invention provides a linker compound of Formula (I) or a salt thereof:

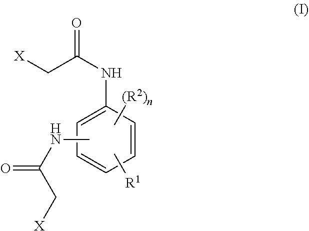

wherein

each X is independently F, CI, Br, or I;

R¹ is H, COOR^{A}, CONH₂, CONHR^{A}, CONR^{A}₂, CONHL, or CONR^{A}L;

L, if present, is a chain terminating in a reactive group R³;

each R^{A}, if present, is independently selected from C₁₋₄alkyl;

n is 0, 1, 2, or 3; and

each R², if present, is independently selected from F, Cl, Me, CF₃, OMe, and OCF₃ or R² is a group as defined for R¹.

Where L is present, the chain is suitably a polyether or polythioether.

Preferably X is Cl, Br or I; more preferably Br or I. This is because bromo and iodo groups are better leaving groups than chloro.

Each R², if present, is independently selected from F, Cl, Me, CF₃, OMe, and OCF₃ or each R² is a group as defined for R¹. Where R² is a group as defined for R¹, preferably n is 1. In other words, the linker compound may have two groups according to the definition for R¹.

Preferably, each R², if present, is independently selected from F, Cl, Me, CF₃, OMe, and OCF₃.

Each R^{A}, if present, is independently selected from C₁₋₄alkyl, preferably methyl or ethyl, most preferably methyl.

The haloacetamide groups are suitably oriented ortho or meta to each other. For example, the linker compound may be a compound of Formula (Ia) or (Ib):

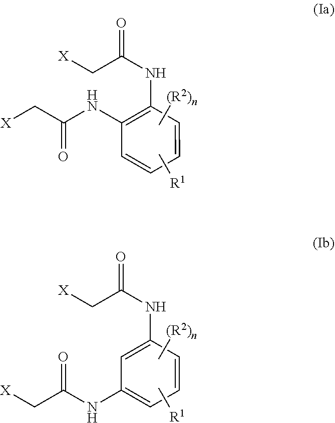

Linker compounds of Formula (Ia) may be termed ortho or 1,2-, while linker compounds of Formula (Ib) may be termed meta or 1,3-.

Preferably, n is 0. That is, R² is not present.

Preferably, in compounds of Formula (Ib) the R¹ group and two haloacetamide groups are arranged in a 1,3,5 configuration.

Accordingly, the linker compound may be a compound of formula (IIa), (IIb) or (IIc):

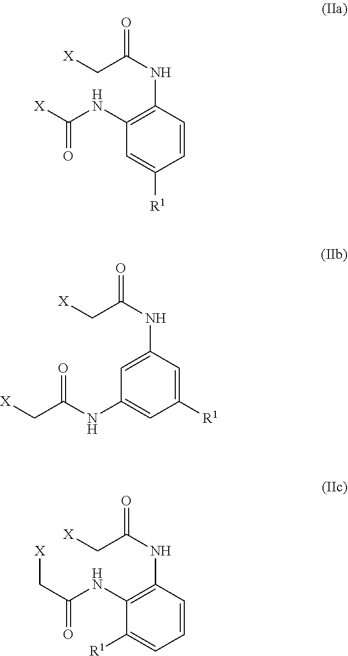

It will be appreciated that, where R¹ is H, compounds of Formula (IIa) and (IIc) are equivalent.

Linker compounds of Formula IIa are referred to as 3,4-subsitituted. Linker compounds of Formula (IIb) are referred to as 3,5-substituted. Linker compounds of Formula (IIc) are referred to as 2,3-substituted. These designations are typically used where R¹ is not H.

In the linker compounds, R¹ is H, COOR^{A}, CONH₂, CONHR^{A}, CONR^{A}₂, CONHL, or CONR^{A}L; L, if present, is a spacer Sₚ terminating in a reactive group R³ (—Sₚ—R³); and each R^{A}, if present, is independently selected from C₁₋₄alkyl, preferably methyl or ethyl, most preferably methyl.

In preferred compounds of the invention, R¹ is H, COOR^{A}, CONH₂, or CONHL; more preferably H, COOMe, CONH₂, or CONHL.

The Group L

L is a chain terminating in a reactive group. In this way, additional functionality can be introduced into the conjugate through reaction at the reactive group. The reactive group may be termed R³.

In other words, L may be —Sₚ—R³, where Sₚ is a spacer and R³ is a reactive group.

The spacer Sₚ may be a polyether or thioether, for example, of up to 32 atoms in length. For example, the spacer S may be a polyether or thioether of 10 to 32 atoms in length, for example 10 to 23 atoms in length.

For example, the spacer Sₚ may be a polytheylene glycol. Accordingly, in some embodiments, the spacer Sₚ is a polyether of formula:

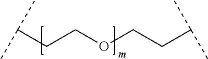

where [symbol] is a point of attachment.

In other words, the spacer may be a PEG chain. m may be selected from 2, 3, 4, 5, 6, 7, 8, 9, and 10. In some cases, m is selected from 3, 4, 5, 6, 7, 8, 9, and 10. In some cases, m is selected from 3, 4, 5, 6, and 7. Spacers having m is 3 and m is 7 are exemplified herein and may be preferred.

R³ is a reactive group. R³ may be a head group of Formula (H) or any group that can be subsequently displaced or modified for the purpose of attaching another molecule. The term head group, as used herein, refers to an aryl bis(2-haloacetamido)-moiety according to Formula (H), or the corresponding portion of any compound of Formulae (I) to (VII).

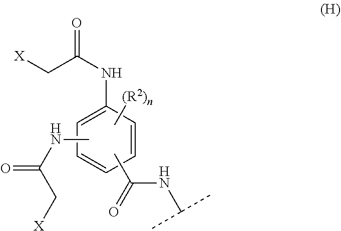

where X, R² and n are as defined above and represents a point of attachment.

The options and preferences for Formula (I) above apply to Formula (H). Accordingly R³ may be a moiety according to (Hi) or (Hii):

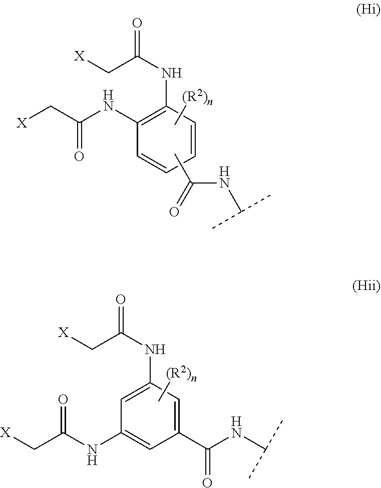

Preferably X is Br or I. Preferably n is 0. Preferably the arrangement in (Hi) is 1,3,4 (plus any R² groups).

Where the linker compound includes two arylene bis haloacetamide head groups joined by a spacer Sₚ the arylene bis haloacetamide head groups may be the same or different.

In other words, or may be Formula (H) or any group that can be subsequently displaced or modified for the purpose of attaching another molecule. For example, R³ may be —N₃ (azide), alkynyl (e.g. —C≡C—H or other group containing a C≡C bond, e.g. a dibenzocyclooctyne (DBCO) substituent), a protected amine (for example an NHP group such as NHBoc, NHFmoc, NHCbz), a leaving group or a moiety of Formula (H); preferably R³ is N₃ (azide), alkynyl (e.g. —C≡C—H), or a moiety of Formula (H).

Suitable leaving groups will be apparent to the skilled person and may include, for example, —I, —Br, —Cl, —OH, —OP, —O-aryl (e.g. a phenoxide leaving group), —OTs, —OMs, —SP, —S—C₁₋₄alkyl, —OSO₂—C₁₋₄alkyl.

P is a protecting group. Suitable protecting groups are known in the art and described in, for example, Greene's Protective Groups in Organic Synthesis (4^{th} Ed) (Peter G. M. Wuts, Theodora W. Greene), the contents of which are incorporated by reference.

In some embodiments, R³ is —N₃ or a group containing a C≡C bond, for example —C≡C—H or DBCO. In these cases, the compounds are suitable for reaction by click chemistry using methods known in the art. —N₃ and DBCO may be preferred because click chemistry can be achieved without the use of a copper catalyst.

In some embodiments, R³ is —N₃. The azide group may be used in click chemistry reactions, or may be reduced to unmask an amine.

Accordingly, the linker compound may be a compound of Formula (III) (when n is not 0, n R² groups may be present as in Formula (I); preferably n is 0):

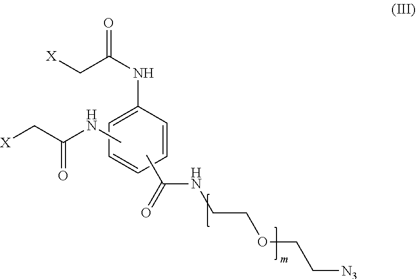

For example, the linker compound may be a compound of Formula (IIIa) or (IIIb) (when n is not 0, n R² groups may be present as in Formula (I); preferably n is 0):

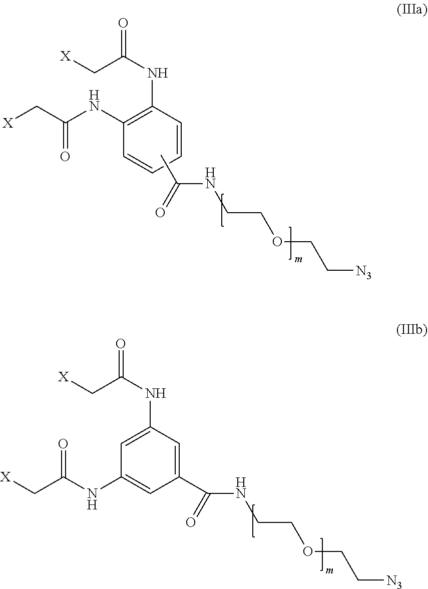

Preferably, the regio-chemical arrangement in Formula (IIIa) is 1,3,4.

In some embodiments, R³ is a moiety of Formula (H). All preferences and options described above for Formulae (I) to (VII) equally apply.

Accordingly, the linker compound may be a compound of Formula (IVa), (IVb) or (IVc) (when n is not 0, n R² groups may be present as in Formula (I); preferably n is 0):

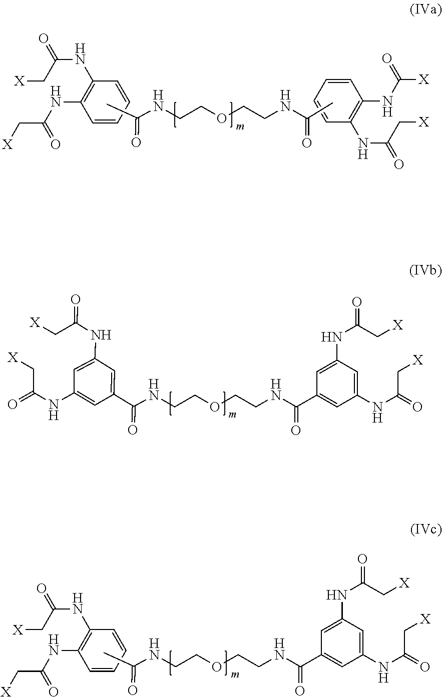

Preferably, the regio-chemical arrangement in Formula (IVa) and (IVc) (left hand head group) is 1,3,4.

X groups may be the same or different. In some embodiments, each X is Br or I. In some embodiments the two X one arylene are Br and the two X or the other arylene are I.

Compounds of formula (IV) may be useful in the preparation of thio-bridged fAb conjugates which can then be conjugated to a further moiety, for example an antibody as shown in Example 10.

In some embodiments L is not present. In other words, R¹ is selected from H, COOR^{A}, CONH₂, CONHR^{A}, and CONR^{A}₂; preferably R¹ is selected from H, COOR^{A}, and CONH₂; most preferably R¹ is selected from H, COOMe, and CONH₂.

In some embodiments, R¹ is H. Accordingly, the linker compound may be a compound of Formula (Va) or (Vb):

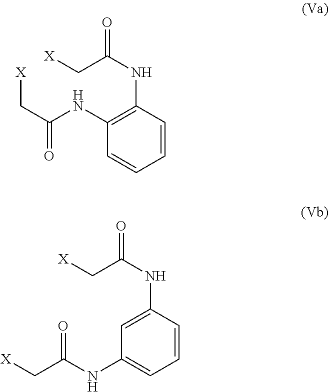

In some embodiments, R¹ is COOR^{A}. Accordingly, the linker compound may be a compound of Formula (VIa), (VIb) or (VIc) (when n is not 0, n R² groups may be present as in Formula (I); preferably n is 0):

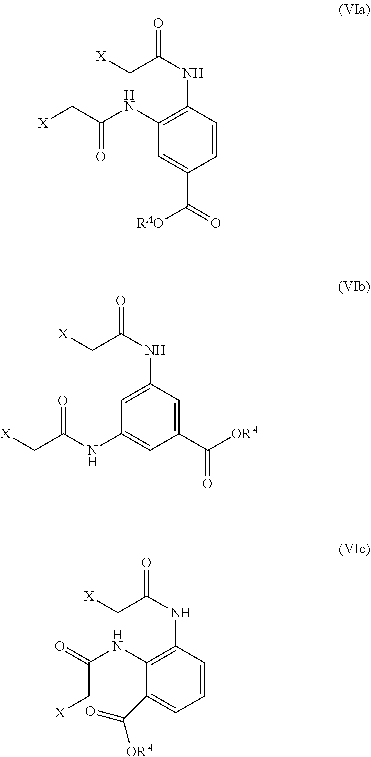

Preferably, R^{A} is Me.

In some embodiments, R¹ is CONH₂, CONHR^{A}, or CONR^{A}₂ (all represented by CONR₂) in the figure below. Accordingly, the linker compound may be a compound of Formula (VIIa), (VIIb) or (VIIc) (when n is not 0, n R² groups may be present as in Formula (I); preferably n is 0):

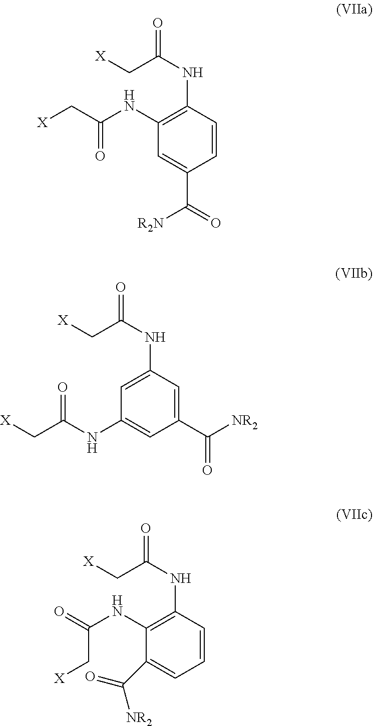

In some embodiments, each X in a compound of any formula described herein is chloro.

In some embodiments, each X in a compound of any formula described herein is bromo.

In some embodiments, each X in a compound of any formula described herein is iodo.

Exemplary Linker Compounds of the Invention

Exemplary linker compounds 1 to 18 are shown below. Compounds 1, 2, 3, 4, 5, 6, and 7 are methyl ester substituted compounds of Formulae (VIa), (VIb), and (VIc). Compounds 8, 9, 10, 11, 12 and 13 are compounds having R¹ is H. These are compounds of Formulae (Va) and (Vb).

Compounds 14, 15, 16, 17 and 18 include a polyethylene glycol (PEG) chain pendant from the aryl ring. This corresponds to the spacer Sₚ. This spacer may terminate in a second aryl bis(2-haloacetamido) moiety when used (e.g. compound 18) which is a compound of Formula (IVa) or may terminate in a different group which is suitable for further reaction which is itself less reactive to thiol displacement that the halogens of the haloacetamide groups (referred to herein as R³). In this latter type, the compound can be considered orthogonally activated. The aryl bis(2-haloacetamido) group may form a bridge between two thiol groups and the spacer terminal group then reacted with another compound, or unmasked and further reacted, to bind a further thiol group of an antibody, antibody fragment, drug, and/or other protein or a different function group of an antibody, antibody fragment, drug and/or other protein. In compounds 14, 15, 16, and 17 the terminal group is an azide and the compounds are compounds of Formula (IIIa) and (IIIb).

In some embodiments, the linker compound of the invention is selected from:

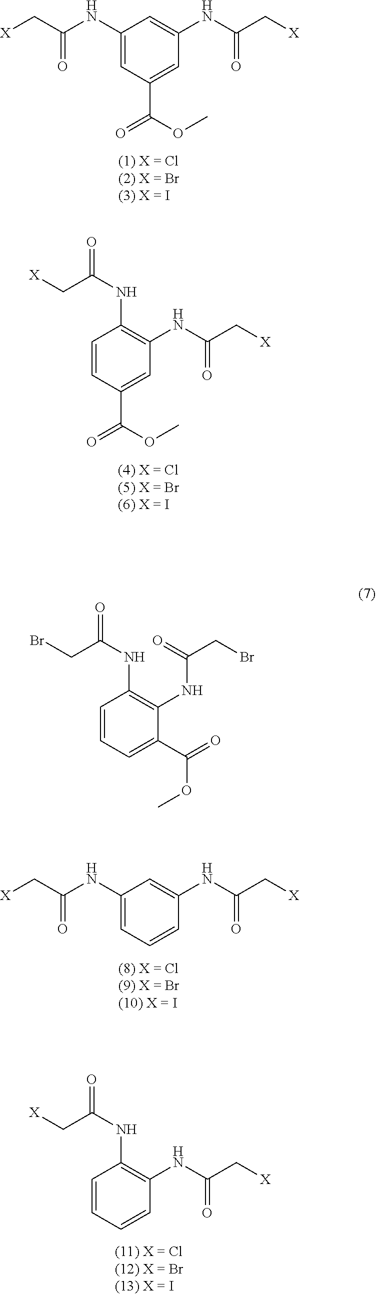

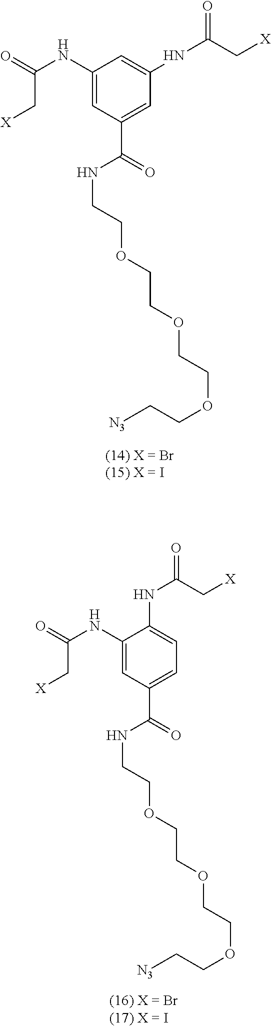

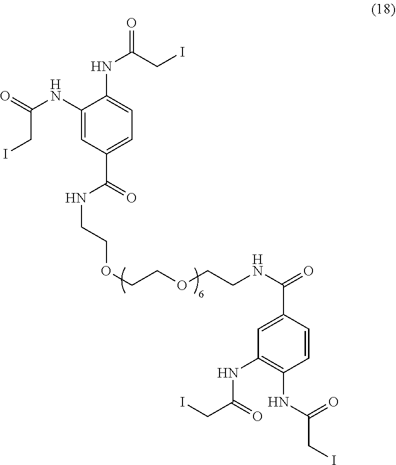

Conjugates

In a further aspect, the invention may provide antibodies, half-antibodies or antibody fragments bearing a linker as described herein. These may be termed antibody conjugates, half-antibody conjugates or antibody fragment conjugates.

The linker typically bridges a two thiols generated by reduction of disulfide bridges in the antibody structure. The bridge may be a heavy-light chain bridge, a heavy-heavy chain bridge, or the linker may form a bridge intra on the same heavy chain.

Accordingly, the present invention may provide a conjugate comprising a motif according to Formula (VIII), where indicate points of attachment to the antibody chain:

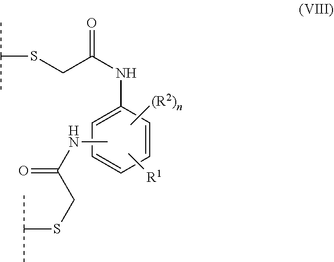

wherein all substituents are as described herein and the linker may optionally be as drawn in any of Formulae (I) to (VII). The preferences for each of those structures equally apply.

The motif may be considered a residue of a linker compound as described herein; that is, the structure of the linker compound than remains after reaction. In other words, the “linker” or “residue” is the portion remaining after displacement of the halide leaving groups. In any conjugate of the invention, in some embodiments the linker is a residue of any one of exemplary compounds 1 to 18.

FIG. 1 shows schematically certain conjugate structures of the invention. FIG. 1(1) shows a half-antibody having a linker rebridging the heavy and light chains and a second (identical) linker linking the heavy chain disulfides intra, rather than inter. FIG. 1(3) shows a half-antibody conjugate in which the linkers are different. This may be referred to a hetero-di-functionalised half-antibody. FIG. 1(2) shows a conjugate which is a whole antibody having a single linker bridging heavy-light chains. If that linker bears a chain terminating in a reactive group (defined as “L” in the claims) then that linker can be orthogonally activated to allow further cross-coupling, for example to generate a tri-specific antibody or antibody-protein conjugate. See FIG. 1(4) and FIG. 1(8). It will be understood that the linker head group moieties may be the same or different. FIG. 1(5) is chemically identical to the structure shown in FIG. 1(1), but represents the physical state in which the half-antibody may be observed. FIG. 1(6) is chemically identical to the structure shown in FIG. 1(3), but again represents the physical state in which the half-antibody may be observed. FIG. 1(7) shows a conjugate which is a whole antibody having two linkers bridging heavy-light chains.

In some embodiments, the invention may therefore provide a product which comprises an antibody having a linker according to Formula (VIII) or any linker as described herein.

In some embodiments, the invention may therefore provide a product which comprises a half-antibody having a linker to Formula (VIII) or any linker as described herein. In other words, the invention may provide a half-antibody having a linker according to Formula (VIII) or any linker as described herein bridging intra two sulfide moieties on the same heavy chain.

In some embodiments, the invention provides a half-antibody having a linker according to Formula (VIII) as defined above bridging intra the two sulfide moieties on the heavy chain, and a further linker according to Formula (VIII) bridging the heavy and light chains. The linkers of Formula (VIII) may be the same (as in FIG. 1(1)) or different (as in FIG. 1(3)).

In some embodiments, the invention may therefore provide a product which comprises an antibody fragment having a linker according to Formula (VIII) or any linker as described herein.

In some embodiments the invention is directed to tri-specific antibodies. In this way specific Fab-Mab conjugates can be produced (see (4) in FIG. 1).

Accordingly, in some embodiments the invention provides a thiobridged Fab. In other words, a Fab fragment rebridged by a linker compound according to Formula (VIII) or any linker as described herein. Preferably the linker compound includes L. For example it may be a linker compound of Formula (IIIa), (IIIb), (IVa), (IVb) or (IVc).

In other words, the invention may provide a thiobridged Fab in which the bridge is of Formula (IXa), (IXb), or (IXc) where represents a point of attachment to the protein chain.

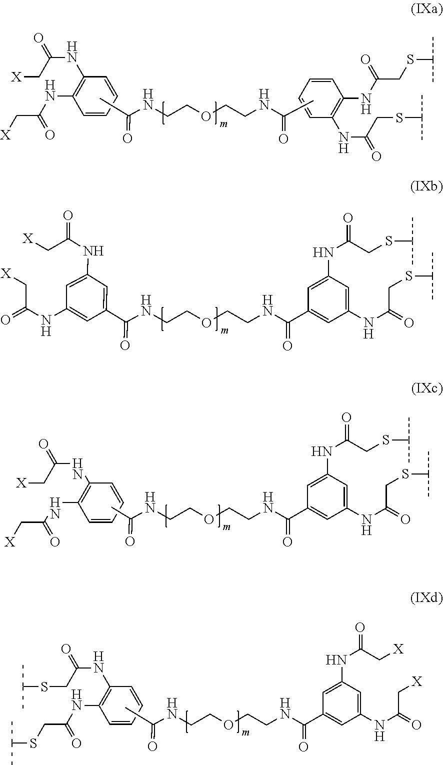

where m is selected from 2, 3, 4, 5, 6, 7, 8, 9, and 10. In some cases, m is selected from 3, 4, 5, 6, 7, 8, 9, and 10. In some cases, m is selected from 3, 4, 5, 6, and 7. m is 3 or m is 7 may be preferred.

In some embodiments, the invention may provide an antibody conjugate, half-antibody conjugate or antibody fragment conjugate (e.g. a thiobridged Fab) which is attached to a drug and/or protein or other compound (e.g. a fluorescent or radio label). Such compounds may be termed products herein. For further discussion of method of making products of the invention, see below. Accordingly, in some embodiments the invention provides a product in which the linker compound includes L, and R³ is replaced by an antibody, half-antibody, antibody fragment, protein, polypeptide, drug or a fluorescent or radio label.

In some embodiments the invention provides a product which comprises a linker motif as shown in Formula (IXa) above, wherein the X groups are replaced by bonds to an antibody, half-antibody, antibody fragment, protein, polypeptide, drug or a fluorescent or radio label. For example, each X may be replaced by resulting from displacement of the X leaving groups by thiol groups of a antibody, half-antibody, antibody fragment, protein, or polypeptide.

Methods

In further embodiments, the invention may provide methods of making conjugates and products comprising conjugates as described herein.

Accordingly, in a further aspect the present invention relates to methods of forming conjugates, the methods comprising treating an antibody or half-antibody or antibody fragment with a reducing agent such as TCEP to effect reduction or partial reduction, and then treating the reduced or partially reduced antibody or half-antibody or antibody fragment with a linker compound according to any Formula described herein to obtain the corresponding conjugate.

Suitably, after a reduction step the reaction is quenched before the addition of the linker compound. This prevents reaction of the linker compound with any residual TCEP. A suitable quenching agent is penta-PEG azide as described in Kantner et al. [2017].

Antibody Conjugates

In some embodiments, the invention relates to methods for producing a mono-functionalised antibody as depicted in FIG. 1(2). That is, a conjugate bearing a single linker bridging a heavy chain and a light chain. This can be achieved by:

(i) partial reduction of an antibody with a reducing agent such as TCEP followed by treatment with a linker compound according to any one of Formulae (I) to (VII) as described herein; or

(ii) exploiting the selectivity of certain linker compounds of the invention for preferential rebridging of the heavy chain-light chain.

Accordingly, the invention may provide a method of producing a mono-functionalised antibody having a motif as depicted in Formula (VIII) or any linker as described herein, the method comprising treating a partially reduced antibody with a linker compound according to any one of Formulae (I) to (VII) as described herein. In some embodiments, the method further comprises the step of providing a partially reduced antibody by reducing an antibody using about 1 to 1.1 equivalent of a reducing agent such as TCEP, for example about 1.1 equivalents. Such a product is shown in FIG. 1(1).

In some embodiments, methods of the invention may provide a method of producing a di-functionalised antibody having two motifs, each as depicted in Formula (VIII) or any linker as described herein, the method comprising treating a partially reduced antibody with a linker compound according to any one of Formulae (I) to (VII) as described herein. At least two equivalents of linker compound will be used. Typically, the linker compound will be provided in excess of two equivalents. In some embodiments, the method further comprises the step of providing a partially reduced antibody by reducing an antibody using about 2 to 2.2 equivalent of a reducing agent such as TCEP, for example about 2.2 equivalents.

In some embodiments, the linker compound is a compound wherein R¹ is COOR^{A} or H. As noted above, the inventors have observed that for light chain-heavy chain bridging, unsubstituted linkers may be preferred. Accordingly, in some methods the compound is a compound of Formula (Va) or (Vb), preferably (Va). That is, R¹ is H and n is 0. Preferably X is bromo. Accordingly, the linker may be selected from compounds 9 and 12, preferably 12.

In other embodiments, the linker compound is a compound in which R¹ is COOR^{A}, preferably COOMe. Accordingly, in some methods the compound is a compound of Formula (VIa), (VIb) or (VIc), preferably (VIa). That is, R¹ is COOMe and n is 0. Preferably X is bromo. Accordingly, the linker may be selected from compounds 2 and 5, preferably 5.

In some embodiments, R¹ comprises L. For example R¹ may be CONHL, as in linker compounds 14, 15, 16, 17, and 18; preferably 14, 15, 16, and 17 owing to the orthogonal functionalisation which can be unmasked and conjugated to a drug moiety, further antibody, half antibody or antibody fragment, a protein, polypeptide, drug or other compound. The method may therefore further comprise a step of attaching a further moiety, for example an antibody, half-antibody, antibody fragment, protein, polypeptide, drug or other compound (e.g. a fluorescent or radio label). In such a method, structures as shown in FIG. 1(4) and FIG. 1(8) may be generated, FIG. 1(4) demonstrating the obtained product when the two head groups are the same, FIG. 1(8) demonstrating the obtained product when the two head groups are different.

In some embodiments, the method for producing a mono-functionalised antibody as depicted in FIG. 1(2) begins with a selectively partially reduced antibody. These methods exploit the preference of certain linker compounds of the invention, in particular linker compounds ortho compounds of Formula (Ia), for rebridging the heavy-light chains. X may be selected from Br and I, preferably X is Br.

Accordingly, in some embodiments the invention provides a method for producing a mono-functionalised antibody having a motif as depicted in Formula (VIII) or any linker as described herein, the method comprising treating a fully reduced antibody with about 1 to 1.1 equivalents of a linker compound of Formula Ia. In some embodiments, X is Br.

Preferably the linker compound is a compound of Formula (IIa).

In some embodiments, the linker compound is a compound wherein R¹ is COOR^{A} or H. As noted above, the inventors have observed that for light chain-heavy chain bridging, unsubstituted linkers may be preferred. Accordingly, in some methods the compound is a compound of Formula (Va). That is, R¹ is H and n is 0. Accordingly, the first linker may be compound 12.

In other embodiments, the linker compound is a compound in which R¹ is COOR^{A}, preferably COOMe. Accordingly, in some methods the compound is a compound of Formula (VIa). That is, R¹ is COOMe and n is 0. Accordingly, the first linker may be compound 5.

In some embodiments, R¹ comprises L. For example R¹ may be CONHL, as in linker compounds 16, 17 and 18, preferably 16 or 17 owing to the orthogonal functionalisation which can be unmasked and conjugated to a drug moiety, further antibody, half antibody or antibody fragment, or a protein. The method may therefore further comprise a step of attaching a further moiety, for example an antibody, half-antibody, antibody fragment, protein, polypeptide, drug or other compound (e.g. a fluorescent or radio label).

Half-Antibody Conjugates

In some embodiments, the method produces a half-antibody having a linker motif bridging intra- the sulfide moieties in the same heavy chain (examples are shown in FIGS. 1(1) and (3)).

Accordingly, in some embodiments the invention provides a method of producing a half-antibody having a motif according to Formula (VIII) or any linker as described herein bridging intra-HC-HC cysteine residues of the hinge region (a so-called half-antibody conjugate), the method comprising treating a fully reduced antibody with a linker compound according to any one of Formulae (I) to (VII) as described herein. Such a half-antibody is labelled HC-LC in the figures, see for example, FIG. 5.

The inventors have observed that with an excess (4 or more equivalents) of both ortho and meta compounds as described herein the major product is the half-antibody conjugate bearing two motifs according to Formula (VIII) or any linker as described herein. Accordingly, the half-antibody product includes two identical linkers, one bridging intra- the sulfide moieties in the heavy chain and the second bridging the heavy and light chains, as shown in FIG. 1(1).

Fewer side products are observed with the ortho compounds. Accordingly, the linker compound may preferably be a compound of Formula (Ia), for example a compound of Formula (IIa) or (IIc). Preferably n is 0. The compound may be a compound of Formula (IIIa); (IVa); (Va); (VIa); (VIc); (VIIa) or (VIIc), preferably (IIIa), (IVa), (Va), (VIa) or (VIIa).

Preferably X is Br or I, preferably Br.

In some embodiments the linker compound is selected from compounds 2, 5, 9, 12, 16 and 17, preferably 5 and 12.

The linker compound is provided in a stoichiometry of at least 4 equivalents, for example at least 5 equivalents, for example about 8 equivalents may be provided.

Where the linker compound is provided in a stoichiometry of less than 4 equivalents, for example about 1 to 1.1 or 2 to 2.2 equivalents, the inventors have observed regioselectivity for compounds of the invention. For example, the inventors have observed a clear preference for intra-HC bridging for meta compounds, especially those having X is I.

Accordingly, in some embodiments the invention provides a method of producing a half-antibody having a motif according to Formula (VIII) or any linker as described herein bridging intra-HC-HC cysteine residues of the hinge region, the method comprising treating a fully reduced antibody with a linker compound of Formulae (Ib) described herein.

Preferably n is 0. That is, the linker compound is a compound of Formula (IIb). For example, the linker compound may be a compound of Formula (IIIb), (IVb), (IVc), (Vb), or (VIb); preferably (IIIb), (IVb), (IVc), (Vb), or (VIb).

Preferably X is I or Br, most preferably I.

Preferably R¹ is H, COOMe, CONH₂, or CONHL.

In some embodiments the linker compound is selected from compounds 2, 3, 9, 10, 14 and 15, preferably compounds 3, 10, and 15.

Where at least one of the linker compounds includes L, the method may include a step of attaching a further moiety, for example an antibody, half-antibody, antibody fragment, protein, polypeptide, drug or other compound (e.g. a fluorescent or radio label). Compound 15 is a preferred example.

In some embodiments, the amount of linker compound is about 2 to 2.2 equivalents. The method may then further comprise rebridging the HC-LC disulfides. The rebridging may generate, where possible, disulfide bridges by oxidation or, more preferably by treating the half-antibody with a further linker compound according the invention. In this latter case, the linker compound maybe any linker compound described herein, and may be used in about 1 to 1.1 equivalents or more per half-antibody conjugate; that is, about 2 to 2.2 equivalents or more per fully reduced antibody, to produce a conjugate as depicted in FIG. 1(3).

In some embodiments, the method produces a half-antibody having a linker bridging intra the sulfide moieties in the heavy chain and a different linker bridging the heavy and light chains (as shown in FIG. 1(3)).

Accordingly, in some embodiments the invention provides a method of producing a hetero-bi-functionalised half-antibody having a first linker which is a motif according to Formula (VIII) or any linker as described herein bridging the heavy and light chains and having a second linker which is a motif according to Formula (VIII) or any linker as described herein bridging intra-HC-HC cysteine residues of the hinge region, the method comprising:

(i) treating a partially reduced antibody with a first linker compound according to any one of Formulae (I) to (VII) as described herein to produce a first conjugate; then

(ii) further reducing said first conjugate to produce a reduced conjugate; then

(iii) treating said reduced conjugate with a second linker compound according to any one of Formulae (I) to (VII), wherein the first and second linker compounds are different, to produce said hetero-bi-functionalised half-antibody conjugate.

In some embodiments, one of the first and second linker compound includes a group L. In other words, at least one of the linker compounds may be a compound of Formula (III) (e.g. IIIa or IIIb, preferably IIa) or (IVa), (IVb) or (IVc) (preferably IVb). Preferably the second linker compound includes L, as in Formulae (III), (IIIa), (IIIb), (IVa), (IVb) and (IVc); more preferably (IIb) and (IIIb). For example, the second linker compound may be selected from compounds 14, 15, 16, and 17, preferably 15.

In some embodiments, the first linker compound does not include L. In some embodiments, the first linker compound is a compound wherein R¹ is COOR^{A} or H. The inventors have observed that for light chain-heavy chain bridging, unsubstituted linker compounds may be preferred. Accordingly, in some methods the linker compound is a compound of Formula (Va) or (Vb), preferably (Va). That is, R¹ is H and n is 0. Preferably X is bromo. Accordingly, the first linker compound may be selected from compounds 9 and 12, preferably 12.

Where at least one of the linker compounds includes L, the method may include a step of attaching a further moiety, for example an antibody, half-antibody, antibody fragment, protein, polypeptide, drug or other compound (e.g. a fluorescent or radio label).

In some embodiments, the first linker compound is a compound in which R¹ is COOR^{A}, preferably COOMe. Accordingly, in some methods the linker compound is a compound of Formula (VIa), (VIb) or (VIc), preferably (VIa). That is, R¹ is COOMe and n is 0. Preferably X is bromo. Accordingly, the first linker compound may be selected from compounds 2 and 5, preferably 5.

In some embodiments, the method further comprises the step of providing a partially reduced antibody by partially reducing an antibody using about 2 to 2.2 equivalents of a reducing agent such as TCEP, for example about 2.2 equivalents.

In some embodiments, the step of further reducing said conjugate uses about 2 to 2.2 equivalents of a reducing agent such as TCEP, for example about 2.2 equivalents.

Suitably, after a reduction step the reaction is quenched before the addition of the linker compound using, for example, penta-PEG azide.

In some embodiments the invention provides a method of producing a hetero-bi-functionalised half-antibody conjugate, the method comprising treating a fully reduced antibody with a first linker compound according to Formula (la) wherein X is Br and a second linker compound of Formula (Ib) wherein X is I. The first and second linker compounds may be added contemporaneously. The first linker compound rebridges the heavy and light chains, while the second linker compound bridges intra-HC-HC cysteine residues of the hinge region. The product is shown in FIG. 1(3) where L1 represents the first linker and L2 represents the second linker. In some embodiments, the first linker compound is a compound according to Formula (IIa) and the second linker compound is a compound according to Formula (IIb).

Fab Conjugates

In some embodiments, the invention relates to a method for producing a stable thio-bridged fAb conjugate. That is, a product comprising a light chain and a digested (truncated) heavy chain linked by a linker of Formula (VIII) as described herein.

fAb starting materials can be commercially obtained, or obtained by digestion of antibodies using methods described in the art, for example using papain. See Andrew & Titus [2000].

The method of producing a thio-bridged fAb conjugate may comprise:

(i) reducing a fAb fragment to produce a reduced fAb precursor; then

(ii) treating said reduced fAb precursor with a linker compound according to any one of Formulae (I) to (VII) as described herein to produce said thio-bridged fAb conjugate.

Preferably, the linker compound includes an L group for further functionalisation/conjugation. That is, R¹ comprises L. For example R¹ may be CONHL, as in linker compounds 14, 15, 16, 17, and 18, preferably compound 18.

In other words, the linker compound may be a compound of Formula (III) (preferably IIIa), (IVa), (IVb) or (Vc) (preferably VIa). Most preferably, the linker compound is a compound of Formula (IVa), (IVb), or (IVc), preferably (IVa). X may be Br or, preferably X is Br.

The method may therefore further comprise treating a reduced or partially reduced antibody, half-antibody, antibody fragment, protein, polypeptide, drug or other compound with said thio-bridged fAb conjugate to form a conjugated product, for example a fAb-mAb or fAb-protein product.

The inventors have observed that for methods described herein selectively is improved when the pH of the reaction is about 7.5. Accordingly, in some methods described herein the pH is below about 8, preferably between about 6 and about 8, most preferably about 7.5. Preferred pH values have been observed to provide higher yields. The inventors note that the reaction goes further to completion, possibly a result of hydrolysis of reagent at high pH and lower reactivity at low pH (due to thiol protonation).

In any method described herein, in some embodiments the antibody is selected from trastuzumab and rituximab.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows representative diagrams of certain conjugate products according to the invention, where the labels L1 and L2 denote head groups rebridging or intra bridging at the disulfides and the black curve indicates a spacer. Native disulfides are indicated by a black line joining black dots.

FIG. 2 shows a comparison of the aqueous stability of methyl ester derivatised 3,4- and 3,5-linker compounds.

FIG. 3 shows a comparison between the aqueous stability of ester and amide derivatised aryl bis-haloacetamide linkers.

FIG. 4 shows the percentage remaining of the bis-haloacetamide derivatives 14-17 in the presence of glutathione (2.2 equiv.) in aqueous phosphate buffer (100 mM, pH 7.5).

FIG. 5 shows SDS-PAGE analysis of cross-linking of fully reduced Tmab with excess of linker compounds 2, 5, 8 and 12 under varying stoichiometry. For key see Example 4.

FIG. 6 shows SDS-PAGE analysis of cross-linking of fully reduced Tmab with excess of linker compounds 2, 5, 8 and 12 at varying pH. For key see Example 5.

FIG. 7 shows SDS-PAGE analysis of cross-linking of fully reduced Tmab with linker compounds 3, 6, 10 and 13. For key see Example 6.

FIG. 8 shows SDS-PAGE analysis of cross-linking of fully reduced Tmab with linker compounds 14, 15, 16 and 17. For key see Example 7.

FIG. 9 shows SDS-PAGE analysis of cross-linking of fully reduced Rituximab with linker compounds 3, 6, 9 and 12. For key see Example 8.

FIG. 10 shows SDS-PAGE analysis of cross-linking of partially reduced Tmab with compounds 2, 5, 9 and 12. For key see Example 9.

FIG. 11 shows SDS-PAGE analysis of bifunctional cross-linking of using sequential method with linker compounds 5 and 15 (a) and a schematic representation of the product (b). For key see Example 9.

FIG. 12 shows (a) a schematic representation of a thio-bridged Fab derivative, (b) SDS-PAGE analysis of conjugation of partially reduced Tmab with Ipilimumab-fAb (for key see Example 10), and (c) a schematic representation of a tri-specific mAb according to the invention. For key see Example 12.

FIG. 13 shows (a) SDS-PAGE analysis of cross-linking of fully reduced Tmab with compounds 15 and 17, (b) characterization of the reaction products from 15 using mass spectroscopy, and (c) characterization of the reaction products from 17 using mass spectroscopy. For key see Example 11.

FIG. 14 shows confocal microscopy of spheroid models.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Compound Stability

The linker compounds of the invention have desirable stability in water, and do not readily undergo hydrolysis.

Significant differences are observed in the aqueous stability of the methyl bis(2-haloacetamido)-benzoate compounds stored in phosphate buffer at pH=7. In general, the 3,5-compounds (meta arrangement) were much more stable than the 2,3- and 3,4-compounds (ortho arrangement). Interestingly, stability did not directly correlate with the halide leaving group within each group of compounds. For 3,4-substituted benzoates, stability was in the order I<Br<Cl (most stable). For 3,5-substituted benzoates, stability was in the order Br<Cl<I (most stable).

Interestingly, when the aryl bears an amido group (that is, R¹ is an amide) the iodo is more stable than the corresponding bromo compound, reversing the usual trend for halide stability.

For compounds having R¹ amide groups, the stability of the ortho and corresponding meta compounds was more similar. Exchanging the ester functional group for an amide functional group reversed the trend for halide stability (iodo- more stable than bromo-) and 2,3-modified were of similar stability to 1,3-modified. The presence of group L improves solubility.

Unsubstituted compounds (R¹ is H) showed the same stability trend as the ester derivatives, but were generally less stable. Without wishing to be bound by any particular theory, the inventors the inventors believe that the presence of an inductively electron-donating/mesomerically electron-withdrawing group R¹ may increase stability. Consequently, in some compounds, conjugates and method described, R¹ is not H.

All are sufficiently stable for use.

Reactivity Towards Thiols

Relative reactivities of the compounds were evaluated towards the small thiol containing peptide glutathione (pH=7, RT). The bis-(2-chloroacetamides) show significantly slower reaction rates than bromo- or iodo-derivatives. The 3,4-bis-Cl compound (4) was found to react significantly faster than the 3,5-bis-Cl compound (1). Without wishing to be bound by any particular theory, the inventors speculate that the difference in reactivity and stability of the ortho and meta compounds may be attributed to differences in the 3D structures of each compound. Methyl 3,5-bis(2-chloroacetamido)benzoate (1) is predicted to have a planar conformation. Thus, steric hindrance could slow the backside attack of thiolate at the carbon attached to halogen (through an S_{N}2 mechanism). This might explain the greater reactivity of ortho compounds like methyl 3,4-bis(2-chloroacetamido)benzoate (4). Compound (4) is not planar and might afford a more accessible carbon for backside attack.

Accordingly, in some compounds, conjugates and method described herein, the linker compound may be selected such that it is not a compound of Formula (VIb).

Although the chloro-derivatives do react with thiols and are viable linking compounds, the examples described herein primarily use bromo- and iodo-acetamides as the reaction rates are quicker. In other words, preferably X is Br or I.

All are sufficiently reactive to be used in the methods of the invention.

Selectivity of Compounds

For ester and amide substituted compounds and unsubstituted arylene compounds, there is a different pattern of thiol bridging for the ortho- and meta-substituted linker compounds when reacted with Trastuzumab.

When the linker compound is provided in excess the reaction produces a half-antibody product with two linkers attached, one bridging intra-heavy chain (H-H) disulphide. The inventors observed that linker compounds having a meta (ie. 1,3-bis-(2-haloacetamido)-) arrangement produce more side products, with more LC-LC and inter H-H produced. Accordingly, where the antibody is fully reduced and the linker is provided in excess (4 or more equivalents) it may be preferable to use an ortho linker compound of Formula (Ia).

However, the inventors have observed that while ortho give less ‘side-products’ (LC-LC, inter-HC), the meta compounds have preference for the intra-HC attachment and the production of half-antibodies. Accordingly, when a fully reduced antibody is treated with fewer than 4 equivalents of linker it is preferable to use a meta compound (that is, a linker compound of Formula (Ib)) for the production of half-antibody conjugates.

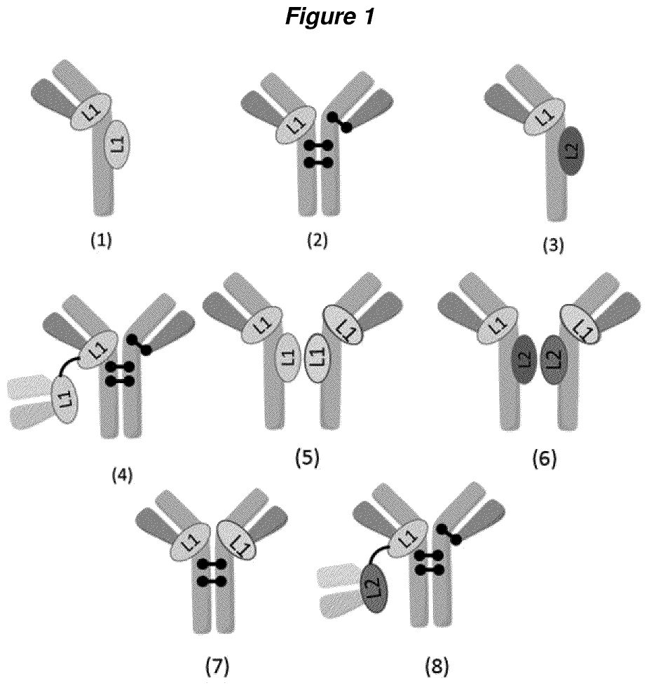

The inventors have also observed that, regardless of regiochemical structure, aryl bis(2-iodoacetamido)-compounds (X is I) show greater preference for intra-HC linking than the corresponding aryl bis(2-bromoacetamido)-compounds (X is Br). Accordingly, for the preferential production of half-antibody conjugates ((1) and (3) in FIG. 1) compounds having X is I are preferred.

For the production of HC-LC conjugates (that is, antibodies having a linker bridging a heavy chain and light chain) the inventors have observed that ortho compounds show greater selectively. Accordingly, where a fully reduced antibody is treated with fewer than 4 equivalents of linker compound it is preferable to use an ortho compound (that is, a linker compound of Formula (Ia)) for the production of monofunctionalised HC-LC antibody conjugates (that is, one linker bridging the heavy and light chains).

The inventors have also observed that, regardless of regiochemical structure, aryl bis(2-bromoacetamido)-compounds (X is Br) show greater preference for HC-LC linking than the corresponding aryl bis(2-iodoacetamido)-compounds (X is I). Accordingly, for the preferential production of HC-LC antibody conjugates ((2) in FIG. 1) compounds having X is Br are preferred.

Where stoichiometric and near-stoichiometric (slight excess) amounts of the reducing agent are used, the linker location in the conjugate may be determined by the regioselectivity of the disulfide bridge reduction.

The inventors have observed that compounds undergo selective reduction of a single HC-LC disulfide bridge, producing one HC-LC conjugate per mAb. For reactions using such a partially reduced antibody, unsubstituted arylene linkers were shown to proceed better. Unsubstituted arylene linkers may be preferred for the production of HC-LC conjugates when less than 4 equivalents of the reducing agent are used.

Example 11 provides a good illustration of this regioselectivity. Fully reduced Tmab (4 disulfides reduced) was treated with only two equivalents of linker compound. The 3,4-compound (X═I) shows high preference for LC-HC, with almost no intra-HC bridging. The 3,5-compound (X═I) shows high preference for intra-HC with almost all HC having one linker, but also forms HC-LC conjugate with 2 linkers (which means the initial intra-HC product with one linker can then further react with another linker to attach the LC). No HC-LC product was observed containing only one linker molecule.

The inventors believe that these preferences extend to other mAbs, with examples relating to rituximab described herein.

It will therefore be recognised that by exploiting the selectivity of the linker compounds it is possible to achieve site-selective monofunctionalisation of mAbs such as Trastuzumab by treating the fully reduced mAb with a limited amount (less than 4 equivalents) of any linker compound of the invention. The invention further offers the potential to achieve hetero-bi-functionalisation of mAbs, either through sequential reduction and treatment with linker compounds or by using mixtures of linker compounds having different site selectivity to contemporaneously hetero-bi-functionalise mAbs.

Stability of Conjugates

Regio-chemistry of the diacetamido compounds has little difference on the stability of the conjugates (antibodies, half-antibodies and antibody fragments comprising linkers of the invention). The 3,4-linked conjugate is slightly more stable than 2,3- and 3,5-linked. All di-acetamido conjugation products were observed to be significantly more stable than the conjugation product of maleimide with glutathione. All conjugates show similar stability when stored in the presence of dithiothreitol (DTT) to samples stored without DTT.

Definitions

Antibody

The term antibody is well understood in the art, and is synonymous with immunoglobulin (Ig). There are five types of antibodies in humans, IgG, IgA, IgM, IgE, and IgD. While the term antibody is intended to cover all types, it will be recognised that in this specification the antibody type will most commonly be an IgG. The inventors observe common features amongst IgG isotypes, for example they have tested IgG1 and IgG4 and observe similar selectivity for the linker compounds and products. It will also be apparent to the skilled person that the term antibody in intended to encompass monoclonal antibodies. The examples use the monoclonal antibodies Trastuzumab, Ipilimumab, and Rituximab. It should be understood that while these monoclonal antibodies may be preferred in some instances, the invention is not intended to be limited to them.

As used herein, and unless context dictates otherwise, the term antibody refers to entire antibody, so for a monomeric form both heavy chains and both light chains in their Y-arrangement.

Half-Antibody

The term half-antibody is understood in the art and used herein to describe a heavy-light chain pair.

Antibody Fragment

As used herein, unless otherwise specified, the term antibody fragment refers to an antigen-binding fragment that can be generated from the variable region of the antibody. The term antibody fragment is therefore intended to encompass F(ab′)₂, Fab, Fab′ and Fv antibody fragments. It will be appreciated that the antibody fragment will be selected depending on purpose, but that selecting and linking the appropriate antibody fragment is within the ability of the skilled person. An especially preferred fragment is a Fab fragment.

In some embodiments the invention is directed to tri-specific antibodies. In this way specific Fab-Mab conjugates can be produced (see (4) and (8) in FIG. 1).

Antibody Conjugate

The term “conjugate” as used herein refers to the presence of a linker according to the invention in product. The linker is attached to an antibody, half-antibody or antibody fragment. The linker may be attached may rebridge two chains, or may link heavy chain disulfides intra- rather than inter.

The linker may attach to a further moiety, for example an antibody, half-antibody, antibody fragment, protein, polypeptide, drug or other compound (e.g. a fluorescent or radio label). For example, linker compounds like linker compound 18 includes two bis(haloacetamido) headgroups which can rebridge thiol bridges and so can be used for the formation of, for example, a mAb protein conjugate (See Example 10), while linker compounds 14, 15, 16, and 17 include an azide which may be used for attaching a further moiety through, for example, click chemistry or amide coupling.

Where a conjugate as described herein is linked to a further moiety, the term “conjugate product” is used. Examples of conjugate products include fAb-mAb conjugates, antibody-drug conjugates, mAb-protein conjuagtes and conjugates bearing labels (e.g. a mAb-fluorophore conjugate).

Protein or Polypeptide

The linkers of the invention may be used to produce, for example, mAb-protein conjugates. It is recognised that the term protein encompasses an antibody, half antibody or antibody fragment, but as used herein the term is intended to encompass non-antibody proteins. The term polypeptide refers to a short chain of amino acids. This can be conjugated via, for example, the azide moiety of linker compounds 14, 15, 16, and 17 (via reduction and peptide coupling) or introduced directly through use of compound 18. Numerous polypeptide drugs are known in the art and may be useful in the methods and products of the present invention.

Abbreviations

It will be appreciated that capitalization of antibody nomenclature varies in the art. The following definitions are not intended to be limited to the combination of upper and lower cases letters used in the following list.

Ab—antibody

Mab/mAb—monoclonal antibody

TCEP—Tris(2-carboxyethyl)phosphine (often supplied as the HCl salt).

Tmab/TmAb—Trastuzumab

IFab/IfAb—Fab fragment of Ipilimumab

Imab/ImAb—Ipilimumab

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### EXAMPLES

### Example 1

### General synthesis of methyl 3,5-bis(2-haloacetamido)benzoates

The methyl 3,5-bis(2-haloacetamido)benzoate linking compounds of the present invention can be made according to the following general synthesis, wherein X is Br or Cl as appropriate.

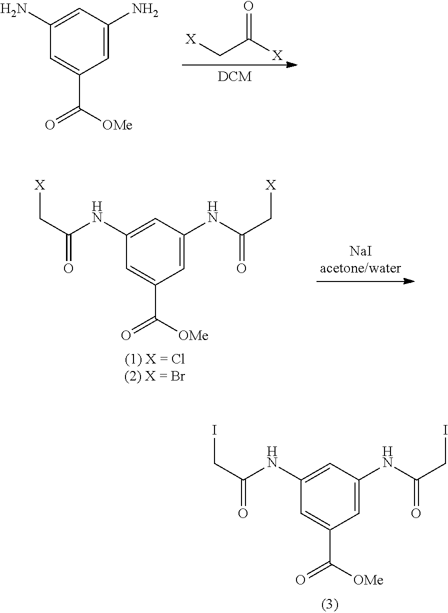

Compound 1

2-Chloroacetyl chloride (1.35 g, 12.04 mmol, 2 eq.) was added dropwise to a cooled solution of methyl 3,5-diaminobenzoate (1 g, 6.02 mmol) in DCM. The mixture was allowed to warm and stirred at room temperature for 2 hrs. The resultant solution was washed with water, saturated ammonium chloride solution, dried over MgSO₄ and the solvent was evaporated under reduced pressure to give the acetamide (1) as pale yellow solid (1.7 g, 89%). ¹H NMR (CDCl₃, 400 MHz): δ=8.31 (s, 2H, NH), 8.20 (s, 1H, Ar), 7.93 (s, 2H, Ar), 4.16 (s, 2H, 2x CH₂), 3.88 (s, 3H, Me). ¹³C NMR (CDCl₃, 100 MHz): δ 165.82, 164.10, 137.58, 131.88, 117.45, 115.56, 52.45, 42.71. ESI-HRMS: Expected for C₁₂H₁₂Cl₂N₂O₄Na (M+Na⁺)=m/z 341.0066. Found: m/z 341.0077.

Compound 2

Methyl 3,5-diaminobenzoate (1.0 g, 6.02 mmol) was dissolved in DMF (10 mL) and bromoacetyl bromide (2.4 eq., 1.26 mL, 14.45 mmol) was added dropwise while stirring over a 10-minute period. Solution was left stirring for 24 hrs and then made up to 150 mL with ethyl acetate and washed with water (4×30 mL) and brine (30 mL) and dried over MgSO₄. The solvent was evaporated under vacuum with pale orange crystals being obtained (2) (1.86 g, 76% yield). ¹H NMR, 400 MHz, CDCl₃: δ 9.48 (s, 1H), 8.1 (s, 1H), 7.9 (s, 2H) 3.88 (s, 4H), 3.77 ppm (s, 3H); ¹³C NMR, 500 MHz, CDCl₃: δ 166.3, 165.0, 138.8, 131.2, 116.5, 115.1, 52.1, 26.8 ppm; HRMS-ESI: calcd for C₁₂H₁₂N₂O₄Br2+: 406.9237; found:406.9261.

Compound 3

KI (1.56 g, 9.43 mmol, 2 eq.) was added to a solution of methyl 3,5-bis(2-chloroacetamido) benzoate (1) (1 g, 3.14 mmol) in dry acetone (20 ml). The mixture was refluxed for 3 hrs. The resultant mixture was filtrated and the solvent was evaporated under reduced pressure to give the acetamide (3) as yellow solid (1 g, 63%). ¹H NMR (CD₃COCD₃, 500 MHz): δ 9.91 (s, 1H), 8.20 (s, 1H), 8.16 (s, 2H), 4 (s, 4H), 3.89 (s, 3H). ¹³C NMR (CD₃COCD₃, 126 MHz): δ 166.82, 166.09, 140.01, 127.53, 131.52, 115.56, 114.00, 51.85, −0.00. HRMS: Expected for C₁₂H₁₃I₂N₂O₄ (M+H+)=m/z 502.8969, Found: m/z 502.8959.

### Synthesis of methyl 3,4-bis(2-haloacetamido)benzoates

The methyl 3,4-bis(2-haloacetamido)benzoate linking compounds of the present invention can be made according to the following general synthesis, wherein X is Br or Cl as appropriate.

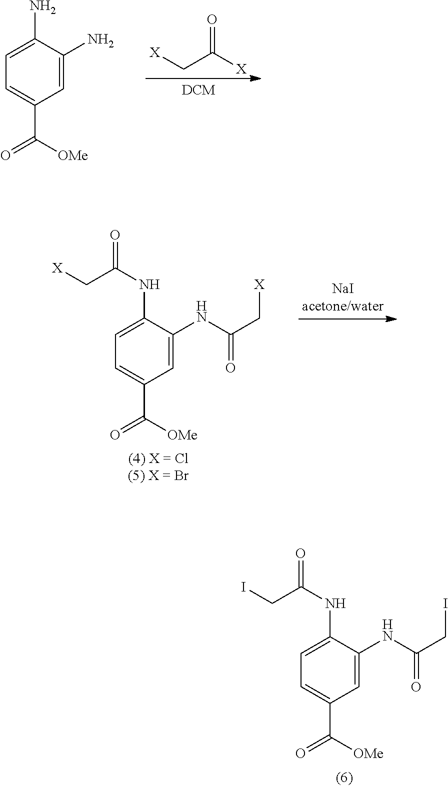

Compound 4

2-Chloroacetyl chloride (1.35 g, 12.04 mmol, 2 eq.) was added dropwise to a cooled solution of methyl 3,4-diaminobenzoate (1 g, 6.02 mmol) in DCM. The mixture was allowed to warm and stirred at room temperature for 2 hrs. The resultant solution was washed with water, saturated ammonium chloride solution, dried over MgSO₄ and the solvent was evaporated under reduced pressure to give the acetamide (X) as bale yellow solid (1.5 g, 79%). ¹H NMR (CDCl₃, 400 MHz): δ 8.84 (s, 1H, NH), 8.54 (s, 1H, NH), 8.04 (d, J=2 Hz, 1H, Ar), 7.94 (dd, J=8.4, 2 Hz, 1H, Ar), 7.75 (d, J=8.4 Hz, 1H, Ar), 4.19 (d, J=12 Hz, 4H, 2 x CH2), 3.88 (s, 3H, Me). ¹³C NMR (CDCl₃, 100 MHz): δ 165.82, 164.10, 137.58, 131.88, 117.45, 115.56, 52.45, 42.71. ESI-HRMS: Expected for C₁₂H₁₂Cl₂N₂O₄Na (M+Na+)=m/z 341.0066. Found: m/z 341.0086.

Compound 5

2-bromoacetyl bromide (2.91 g, 14.44 mmol, 2.4 eq.) was added dropwise to a solution of methyl 3,4-diaminobenzoate (1.0g, 6.02 mmol, 1.0 eq.) in dimethylformamide (DMF) at RT. The reaction mixture was kept under stirring at RT for 24 hours. Approximately 150 mL of ethyl acetate was added to the obtained solution which was washed out with distilled water for 3 times, saturated ammonium chloride for 1 time, dried over MgSO4 and the remaining solvent was evaporated under reduced pressure to afford Methyl 3,4-bis(2-bromoacetamido)benzoate (5) as a yellowish powder (2.40 g, 98%). Further recrystallization with DCM was carried.

¹H NMR (500 MHz, Chloroform-d) δ 8.75 (s, 1H, NH), 8.47 (s, 1H, NH), 8.05 (s, 1H, Ar), 7.98 (d, J=8 Hz, 1H, Ar), 7.77 (d, J=8 Hz, 1H, Ar), 4.07 (s, 2H, CH₂), 4.04 (s, 2H, CH₂), 3.91 (s, 3H, CH₃). ¹³C NMR (100 MHz, CDCl₃) δ 167.79, 167.15, 165.31, 134.72, 128.82, 127.21, 126.40, 124.76, 52.43, 29.11. Esi-HRMS: Expected for C₁₂H₁₂N₂O₄Br₂ (M+H⁺)=m/z 404.9091, Found: m/z 404.9130.

Compound 6

KI (1.56 g, 9.43 mmol, 2 eq.) was added to a solution of methyl 3,4-bis(2-chloroacetamido) benzoate (4) (1 g, 3.14 mmol) in dry acetone (20 ml). The mixture was refluxed for 3 hrs. The resultant mixture was filtrated and the solvent was evaporated under reduced pressure to give the acetamide (6) as yellow solid (1 g, 63%). ¹H NMR (CDCl₃, 500 MHz): δ 9.73 (s, 1H), 9.61 (s, 1H), 8.02 (1H), 7.80 (d, J=8.5 Hz, 1H), 7.65 (d, J=8.5 Hz, 1H), 3.86 (s, 3H), 3.86 (s, 4H). ¹³C NMR (CDCl₃, 126 MHz): δ 167.79, 167.15, 165.94, 127.53, 127.36, 126.65, 124.22, 52.11, −0.74, −1.0. HRMS: Expected for C₁₂H₁₃I₂N₂O₄ (M+H+)=m/z 502.8969, Found: m/z 502.8959.

Compound 7

2,3-diaminobenzoic acid (1.0 g, 6.57 mmol, 1 eq.) was dissolved into 10 ml of anhydrous methanol (MeOH) and acidified to pH=1 through the addition of four drops of concentrated H₂SO₄. Reaction was heated to 50° C. and kept under stirring and reflux for 72 hours. The product was subsequently neutralized with sodium carbonate, and concentrated under reduced pressure. The remaining solution was then washed out with distilled water for 3 times, saturated ammonium chloride for 1 time, dried out with MgSO₄, and excess of solvent was evaporated under reduced pressure to afford Methyl 2,3-diaminobenzoate as a dark brown solid (0.70g, 64%). 2-bromoacetyl bromide (0.58 g, 2.88 mmol, 2.4 eq.) was then added dropwise to a solution of methyl 2,3-diaminobenzoate (0.2 g, 1.20 mmol, 1.0 eq.) in anhydrous DCM at RT. Reaction flask was kept under stirring at RT for 24 hours. The resultant solution was then washed out with distilled water for 3 times, concentrated ammonium chloride for 1 time, dried out with MgSO₄, and excess of solvent was evaporated under reduced pressure to afford methyl 2,3-bis(2-bromoacetamido)benzoate (7) as a yellowish solid (0.38 g, 78%). ¹H NMR (CDCl₃, 500 MHz): δ 10.70 (s, 1H, NH), 9.07 (s, 1H, NH), 7.92 (d, J=8 Hz, 2H, Ar), 7.38 (t, J=8 Hz, 1H, Ar), 4.09 (s, 2H, CH2), 3.99 (s, 2H, CH2), 3.95 (s, 3H, CH2). ¹³C NMR (CDCl₃, 100 MHz): δ 167.33, 166.17, 164.80, 131.99, 128.79, 126.38, 122.61, 52.89, 29.28, 28.61. ESI-HRMS: Expected for C₁₂H₁₂N₂O₄Br₂ (M+H+)=m/z 406.9237, Found: m/z 406.9251.

Compound 8

2-Chloroacetyl chloride (9.3 g, 84 mmol, 6 equiv.) was added dropwise to a cooled aqueous NaOH (0.55 M) solution of benzene-1,3-diamine (1.5 g, 14 mmol). The mixture was allowed to warm and stirred at room temperature overnight. The obtained precipitate was filtered, washed with water 5-6 times and completely dried under high vacuum to give the acetamide (8) as a white solid (0.97 g, 27%). ¹H NMR (DMSO-d₆, 400 MHz): δ 10.34 (s, 2H, NH), 7.96 (t, J=2.0 Hz, 1H, Ar), 7.37-7.25 (m, 3H, Ar), 4.25 (s, 4H, 2 x CH2). ¹³C NMR (DMSO-d₆, 100 MHz): δ 164.61, 138.82, 129.14, 114.81, 110.36, 45.53. ESI-HRMS: Expected for C₁₀H₁₀Cl₂N₂O₂Na (M+Na⁺)=m/z 283.0012. Found: m/z 283.0049.

Compound 9

2-bromoacetyl bromide (2.05 g, 10.2 mmol, 2.2 equiv.) was added dropwise to a cooled solution of benzene-1,3-diamine (0.5 g, 4.6 mmol) and TEA (1.35 g, 13.3 mmol, 2.2 equiv.) in DCM. The obtained precipitate was filtered and washed with water 5-6 times before washing with ether. The solid compound was dried under high vacuum to give the acetamide (9) as a pale yellow solid (2.48 g, 77%). ¹H NMR (DMSO-d₆, 500 MHz): δ 10.42 (s, 2H, 2 X NH), 7.96 (t, J=2.0 1H, Ar), 7.37-7.24 (m 3H, Ar), 4.04 (s, 4H, 2 X CH2). ¹³C NMR (DMSO-d₆, 126 MHz): δ 164.79, 138.95, 129.14, 114.72, 110.17, 30.36. ESI-HRMS: Expected for C₁₀H₁₀Br₂N₂O₂Na (M+Na⁺)=m/z 370.9001. Found: m/z 370.9039.

Compound 10

KI (1.3 g, 7.7 mmol, 4 equiv.) was added to a solution of N,N′-(1,3-phenylene)bis(2-chloroacetamide) (8) (0.50 g, 1.9 mmol) in dry acetone (20 mL). The mixture was refluxed for 3 hs. The obtained mixture was filtrated and the solvent was evaporated under reduced pressure to give the acetamide (10) as a yellow solid (0.63 g, 74%). ¹H NMR (DMSO-d₆, 500 MHz): δ 10.36 (s, 2H, NH), 7.92 (t, J=2.0 Hz, 1H, Ar), 7.33-7.22 (m, 3H, Ar), 3.83 (s, 4H, 2 X CH2). ¹³C NMR (DMSO-d₆, 126 MHz): δ 166.56, 139.18, 129.10, 114.36, 109.86, 1.58. HRMS: Expected for C₁₀C₁₀I₂N₂O₂Na (M+Na⁺)=m/z 466.8724, Found: m/z 466.8758. HPLC: column: HiQ Sil HS (150×4.60 mm). Mobile phase: isocratic: (0.75 mL/min), 35% MeCN: 65% water. Detection at 280 nm. Retention time, 8.89 minutes, purity, 93.4%.

Compound 11

2-Chloroacetyl chloride (9.3 g, 84 mmol, 6 equiv.) was added dropwise to a cooled aqueous NaOH (0.55 M) solution of benzene-1,2-diamine (1.5 g, 14 mmol). The mixture was allowed to warm and stirred at room temperature overnight. The obtained precipitate was filtered, washed with water 5-6 times and the obtained solid compound obtained was completely dried under high vacuum to give the acetamide (11) as a white solid (1.8 g, 51%). ¹H NMR (DMSO-d₆, 400 MHz): δ 9.69 (s, 2H, NH), 7.55 (dd, J=7.4, 3.7 Hz, 2H, Ar), 7.23 (dd, J=6.1, 3.5, 2 Hz, 2H, Ar), 4.34 (s, 4H, 2 x CH2). ¹³C NMR (CDCl₃, 100 MHz): δ 165.12, 130.17, 125.61, 125.08, 43.19. ESI-HRMS: Expected for C₁₀H₁₀Cl₂N₂O₂Na (M+Na⁺)=m/z 283.0012. Found: m/z 283.0010.

Compound 12

2-bromoacetyl bromide (2.05g, 10.2 mmol, 2.2 equiv.) was added dropwise to a cooled solution of benzene-1,2-diamine (0.50 g, 4.6 mmol) and TEA (1.03 g, 10.2 mmol, 2.2 equiv.) in DCM. The obtained precipitate was filtered and washed with water 5-6 times before washing with ether. The solid compound was dried under vacuum to give the acetamide (12) as a yellow solid (1.87 g, 58%). ¹H NMR (DMSO-d₆, 500 MHz): δ 9.71 (s, 2H, NH), 753 (dd, J=7.5, 3.7 Hz, 2H, Ar), 7.23 (dd, J=6.0, 3.5 Hz 2H, Ar), 4.13 (s, 4H, 2 X CH2). ¹³C NMR (DMSO-d₆, 126 MHz): δ 165.14, 130.27, 125.56, 124.93, 30.14. ESI-HRMS: Expected for C₁₀H₁₀Br₂N₂O₂Na (M+Na⁺)=m/z 370.9001. Found: m/z 370.9012.

Compound 13

KI (1.3 g, 7.7 mmol, 4 equiv.) was added to a solution of N,N′-(1,3-phenylene)bis(2-chloroacetamide) (11) (0.50 g, 1.9 mmol) in dry acetone (20 mL). The mixture was refluxed for 3 hs. The obtained mixture was filtrated and the solvent was evaporated under reduced pressure to give the acetamide (13) as a yellow solid (0.63 g, 45%). ¹H NMR (DMSO-d₆, 500 MHz): δ 9.63 (s, 2H, NH), 7.49 (dd, J=7.4, 3.7 Hz, 2H, Ar), 7.20 (dd, J=6.1, 3.5 Hz, 2H, Ar), 3.90 (s, 4H, 2 X CH2). ¹³C NMR (DMSO-d₆, 126 MHz): δ 166.92, 130.42, 125.35, 124.70, 1.49. HRMS: Expected for C₁₀H₁₀I₂N₂O₂Na (M+Na⁺)=m/z 466.8724. Found: m/z 466.8770.

Compound 14

### Synthesis of 3,5-bis(2-chloroacetamido)benzoic acid (19)

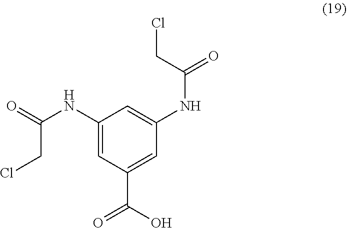 [SMILES: O=C(CCl)Nc1cc(NC(=O)CCl)cc(C(=O)O)c1]

2-Chloroacetyl chloride (1.62 g, 14.5 mmol, 2.2 equiv.) was added dropwise to a cooled solution of 3,4-diaminobenzoate (1.0 g, 6.6 mmol) in THF. The mixture was allowed to warm and stirred at room temperature for 2 hs. The obtained precipitate was filtered, washed with water 5-6 times and then completely dried under high vacuum to give the acetamide (19) as a bale yellow solid (1.8 g, 90%). ¹H NMR (CD₃COCD₃, 400 MHz): δ 9.64 (s, 2H, NH), 8.24 (d, J=78.3 Hz, 3H, Ar), 4.30 (s, 4H, CH₂). ¹³C NMR (CD₃COCD₃, 101 MHz) δ 166.91, 140.07, 132.58, 117.11, 115.48, 44.07. ESI-HRMS: Expected for C₁₁H₉Cl₂N₂O₄ (M−H⁺)=m/z 302.9939. Found: m/z 302.9956.

### Synthesis of 2,5-dioxopyrrolidin-1-yl 3,5-bis(2-chloroacetamido)benzoate (20)

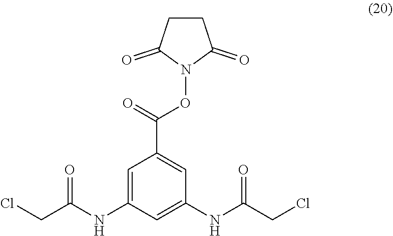 [SMILES: O=C(CCl)Nc1cc(NC(=O)CCl)cc(C(=O)ON2C(=O)CCC2=O)c1]

A solution of EDC.HCl (0.63 g, 3.3 mmol, 1.1 equiv.) in DMF (5 mL) was added to a stirred solution of acid (19) (1 g, 3 mmol) and N-hydroxysuccinimide (0.38 g, 3.3 mmol, 1.1 equiv.) in THF at room temperature. The reaction was then stirred at room temperature for 2 hs before being concentrated under reduced pressure. The obtained residue was dissolved in EtOAc and washed with water, dried over MgSO₄ and the solvent was evaporated under reduced pressure giving a foamy solid. The crude was further purified by precipitation (EtOAc/petroleum ether) to give acetamide the (20) a yellow solid (0.75 g, 57%). ¹H NMR (CD₃COCD₃, 400 MHz,): δ 9.78 (s, 2H, NH), 8.34 (d, J=64.1 Hz, 3H, Ar), 4.32 (s, 4H, 2 x ClCH₂CO), 2.99 (s, 4H, COCH2CH2CO). ¹³C NMR (CD₃COCD₃, 101 MHz): δ 170.86, 170.43, 170.37, 165.95, 162.44, 140.81, 127.32, 117.21, 117.15, 44.06, 26.40. ESI-HRMS: Expected for C₁₅H₁₃Cl₂N₃O₆Na (M+Na⁺)=m/z 424.0074. Found: m/z 424.0106.

### 2-(2-(2-(2-azidoethoxy)ethoxy)ethoxy)ethan-1-amine (22)

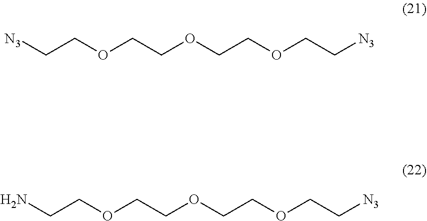 [SMILES: [N-]=[N+]=NCCOCCOCCOCCN=[N+]=[N-] ; [N-]=[N+]=NCCOCCOCCOCCN]

A solution of Ph₃P (0.54 g, 2.1 mol, 1 equiv.) in ether (5 mL) was added dropwise to aqueous HCl (5%, 5 mL) solution of tri-PEG azide (21) (0.50 g, 2.1 mmol), the mixture was left stirring for 24 hs. Then, the ether was removed under reduced pressure, and the aqueous layer was extracted with DCM until Ph₃P oxide was not detected in the aqueous layer. The aqueous layer was adjusted to pH=12, and azide-linked amine was extracted from the aqueous layer with DCM. The combined DCM solution was evaporated under reduced pressure to give the azide-linked amine (22) as a light yellow liquid (0.25 g, 56%). ¹H NMR (CDCl₃, 400 MHz): δ 3.65-3.53 (m, 11 H), 3.44 (td, J=5.2, 1.3 Hz, 2H), 3.41-3.22 (m, 3H), 2.79 (td, J=5.3, 1.4 Hz, 2H). ¹³C NMR (CDCl₃, 101 MHz): δ 73.41, 70.65, 70.60, 70.58, 70.23, 69.95, 50.63, 41.75. ESI-HRMS: Expected for C₈H₁₉N₄O₃ (M+H⁺)=m/z 219.1452. Found: m/z 219.1464.

### N,N′-(5((2-(2-(2-(2-azidoethoxy)ethoxy)ethoxy)ethyl)carbamoyl)-1,3-phenylene)bis(2-chloroacetamide) (23)

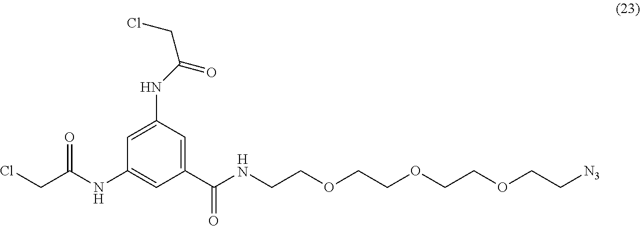 [SMILES: [N-]=[N+]=NCCOCCOCCOCCNC(=O)c1cc(NC(=O)CCl)cc(NC(=O)CCl)c1]

2-(2-azidoethoxy)ethan-1-amine (22) (0.34 g, 1.6 mmol, 1.3 equiv.) was added to an anhydrous solution of activated ester (20) (0.50 g, 1.2 mmol) in THF. The reaction mixture was then stirred at room temperature for 1 h before being concentrated under reduced pressure. The obtained residue was dissolved in DCM and washed with water, dried over MgSO₄ and the solvent was evaporated under reduced pressure. The crude was further purified by silica gel chromatography: (5% to 20% MeOH/DCM) to give azide-linked acetamide (23) as a white solid (0.39 g, 62%). ¹H NMR (CDCl₃, 400 MHz,): δ 8.78 (s, 2H, NHCH), 8.02 (s, 1H, Ar), 7.68 (s, 2H, Ar), 7.20 (d, J=8.8 Hz, 1H, CONHCH2), 4.13 (s, 4H, 2 x ClCH₂CO), 3.75-3.41 (m, 14H), 3.27 (t, J=5.0 Hz, 2H, CH₂CH₂N₃). ¹³C NMR (CDCl₃, 101 MHz): δ 166.77, 164.69, 137.88, 135.96, 115.04, 114.31, 70.57, 70.51, 70.47, 70.24, 69.84, 69.55, 50.57, 42.96, 40.08. ESI-HRMS: Expected for C₁₅H₁₃Cl₂N₃O₆Na (M+Na⁺)=m/z 424.0074. Found: m/z 424.0106. HPLC: column: HiQ Sit HS (150×4.60 mm). Mobile phase: isocratic: (0.75 mL/min), 35% MeCN: 65% water. Detection at 280 nm. Retention time: 6.54 minutes, purity: 99.3%.

### N,N′-(5-((2-(2-(2-(2-azidoethoxy)ethoxy)ethoxy)ethyl)carbamoyl)-1,3-phenylene)bis(2-bromoacetamide) (14)

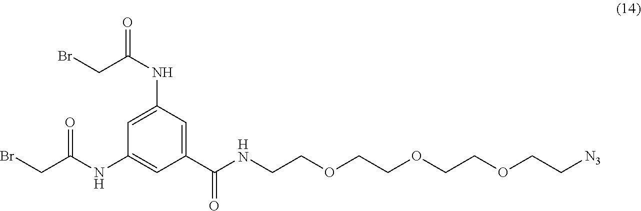 [SMILES: [N-]=[N+]=NCCOCCOCCOCCNC(=O)c1cc(NC(=O)CBr)cc(NC(=O)CBr)c1]

KBr (1.4 g, 12 mmol, 6 equiv.) was added to a solution of N,N′-(4-((2-(2-(2-(2-azidoethoxy)ethoxy)ethoxy)ethyl)carbamoyl)-1,3-phenylene)bis(2-chloroacetamide) (23) (1.0 g, 2.0 mmol) in dry acetone (20 mL). The mixture was refluxed for 4 days. The obtained mixture was filtrated and the solvent was evaporated under reduced pressure. The crude was further purified by silica gel chromatography: (50% to 70% acetone/chloroform) to give the azide-linked acetamide 14 as a yellow solid (0.6 g, 51%). ¹H NMR (CDCl₃, 500 MHz,): δ 8.79 (s, 2H, 2 x NHCH), 7.96 (s, 1H, Ar), 7.67 (s, 2H, Ar), 7.10 (s, 1H, NHCH), 3.95 (s, 4H, BrCH₂CO), 3.84-3.44 (m, 14H), 3.27 (t, J=5.0 Hz, 2H, CHCH₂N₃). ¹³C NMR (CDCl₃, 126 MHz): δ 167.04, 164.62, 138.22, 135.84, 114.87, 114.14, 70.67, 70.59, 70.55, 70.36, 69.94, 69.59, 50.64, 40.24, 29.42. ESI-HRMS: Expected for C₁₉H₂₇Br₂N₆O₆ (M+H⁺)=m/z 593.0353. Found: m/z 593.0344. HPLC: column: HiQ Sil HS (150×4.60 mm). Mobile phase: isocratic: (0.75 mL/min), 40% MeCN: 60% water. Detection at 280 nm. Retention time: 6.52 minutes, purity: 99.4%.

Compound 15

### N,N′-(4-((2-(2-(2-(2-azidoethoxy)ethoxy)ethoxy)ethyl)carbamoyl)-1,3-phenylene)bis(2-iodoacetamide) (15)

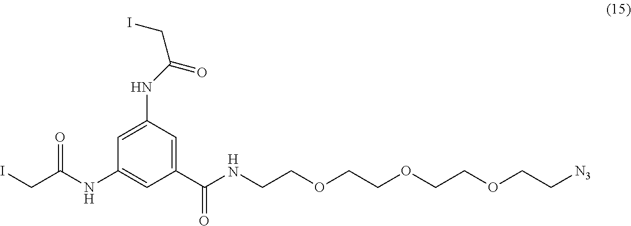 [SMILES: [N-]=[N+]=NCCOCCOCCOCCNC(=O)c1cc(NC(=O)CI)cc(NC(=O)CI)c1]

KI (1.0 g, 8.0 mmol, 4 equiv.) was added to a solution of N,N′-(4-((2-(2-(2-(2-azidoethoxy)ethoxy)ethoxy)ethyl)carbamoyI)-1,3-phenylene)bis(2-chloroacetamide) (23) (1.0 g, 2.0 mmol) in dry acetone (20 mL). The mixture was refluxed for 3 hs. The obtained mixture was filtrated and the solvent was evaporated under reduced pressure. The crude was further purified by silica gel chromatography: (50% to 70% acetone/chloroform) to give azide-linked acetamide (15) as a yellow solid (1.19 g, 88%). ¹H NMR (CD₃COCD₃, 500 MHz): δ 9.98 (s, 2H, 2 x NHCH), 7.93 (d, J=3.7 Hz, 3H, Ar), 7.69 (d, J=5.3 Hz, 1H, CONHCH2), 3.95 (s, 4H, ICH₂CO), 3.60-3.11 (m, 16H). ¹³C NMR (CD₃COCD₃, 126 MHz): δ 170.49, 167.51, 140.48, 137.24, 114.46, 113.41, 71.14, 70.58, 51.42, 40.42, 1.34. ESI-HRMS: Expected for C₁₉H₂₇Cl₂N₆O₆ (M+H⁺)=m/z 505.1364. Found: m/z 505.141600. HPLC: column: HiQ Sil HS (150×4.60 mm). Mobile phase: isocratic: (0.75 mL/min), 35% MeCN: 65% water. Detection at 280 nm. Retention time: 10.31 minutes, purity: 97.8%.

Compound 16

### 3,4-bis(2-chloroacetamido)benzoic acid (24)

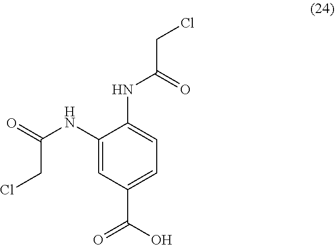 [SMILES: O=C(CCl)Nc1ccc(C(=O)O)cc1NC(=O)CCl]

2-chloroacetyl chloride (1.62 g, 14.5 mmol, 2.2 equiv.) was added dropwise to a cooled solution of 3,4-diaminobenzoate (1.0 g, 6.6 mmol) in THF. The mixture was allowed to warm and stirred at room temperature for 2 hs. The obtained precipitate was filtered, washed with water 5-6 times and the obtained solid compound was completely dried under high vacuum to give the acetamide (24) as a white solid (1.8 g, 90%). ¹H NMR (DMF-ch, 500 MHz): δ 10.09 (d, J=20.4 Hz, 2H, NH), 8.30 (d, J=1.9 Hz, 1H, Ar), 8.00-7.83 (m, 2H, Ar), 4.45 (d, J=7.7 Hz, 4H, CH₂). ¹³C NMR (126 MHz, DMF-d₇): δ 166.86, 166.02, 165.84, 135.31, 129.94, 128.03, 127.15, 127.00, 124.12, 43.64, 43.56. ESI-HRMS: Expected for C₁₁H₁₁Cl₂N₂O₄Na (M+Na⁺)=m/z 305.0090. Found: m/z 305.0092. HPLC: column: HiQ Sil HS (150×4.60 mm). Mobile phase: isocratic: (0.75 mL/min), 35% MeCN: 65% water. Detection at 280 nm. Retention time: 4.38 minutes, purity: 93%.

### 2,5-dioxopyrrolidin-1-yl 3,4-bis(2-chloroacetamido)benzoate (25)

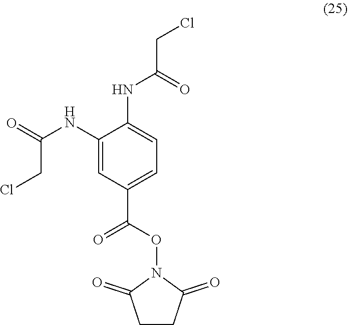 [SMILES: O=C(CCl)Nc1ccc(C(=O)ON2C(=O)CCC2=O)cc1NC(=O)CCl]

A solution of EDC.HCl (0.63 g, 3.3 mmol, 1.1 equiv.) in DMF (5 mL) was added to a stirred solution of acid (24) (1 g, 3 mmol) and N-hydroxysuccinimide (0.38 g, 3.3 mmol, 1.1 equiv.) in THF at room temperature. The reaction mixture was then stirred at room temperature for 2 hs before being concentrated under reduced pressure. The obtained residue was dissolved in EtOAc and washed with water, dried over MgSO₄ and the solvent was evaporated under reduced pressure giving foamy solid. The crude was further purified by precipitation (EtOAc/petroleum ether) to give the acetamide (25) as a yellow solid (0.6 g, 45%). ¹H NMR (CD₃COCD₃, 400 MHz): δ 9.51 (d, J=16.2 Hz, 2H, NH), 8.34 (s, 1H, Ar), 8.18-7.89 (m, 2H, Ar), 4.38 (s, 4H, 2 x ClCH₂CO), 2.98 (s, 4H, COCH2CH2CO). ¹³C NMR (CD₃COCD₃, 100 MHz): δ 170.48, 166.85, 166.31, 161.95, 138.26, 130.66, 129.04, 128.65, 125.21, 122.83, 43.88, 26.39. ESI-HRMS: Expected for C₁₅H₁₃Cl₂N₃O₆Na (M+Na⁺)=m/z 424.0074. Found: m/z 424.0094.

### N,N′-(4-((2-(2-(2-(2-azidoethoxy)ethoxy)ethoxy)ethyl)carbamoyl)-1,2-phenylene)bis(2-chloroacetamide) (26)

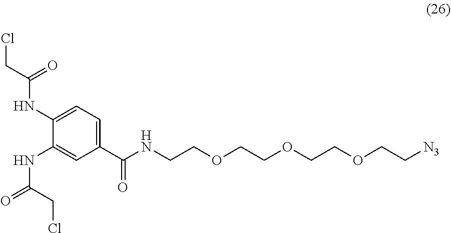 [SMILES: [N-]=[N+]=NCCOCCOCCOCCNC(=O)c1ccc(NC(=O)CCl)c(NC(=O)CCl)c1]

2-(2-azidoethoxy)ethan-1-amine (22) (0.34 g, 1.6 mmol, 1.3 equiv.) was added to an anhydrous solution of activated ester (25) (0.50 g, 1.2 mmol) in THF. The reaction mixture was then stirred at room temperature for 1 h before being concentrated under reduced pressure. The obtained residue was dissolved in DCM and washed with water, dried over MgSO₄ and the solvent was evaporated under reduced pressure. The crude was further purified by silica gel chromatography: (5% to 20% MeOH/DCM) to give the azide-linked acetamide (26) as a white solid (0.25 g, 40%). ¹H NMR (CDCl₃, 500 MHz): δ 9.05 (s, 2H, 2 x NHCH), 7.66 (s, 1H, Ar), 7.58-7.40 (m, 2H, Ar), 7.14 (t, J=5.4 Hz, 1 H, CONHCH₂), 4.15 (d, J=10.6 Hz, 4H, 2 x CICH₂CO), 3.69-3.46 (m, 14H), 3.26 (t, J=5.0 Hz, 2H, CH₂CH₂N₃). ¹³C NMR (CDCl₃, 126 MHz): δ 166.29, 166.09, 165.49, 132.96, 132.74, 129.15, 125.57, 125.10, 124.86, 70.63, 70.60, 70.51, 70.24, 69.94, 69.65, 50.63, 42.91, 42.68, 40.01. ESI-HRMS: Expected for C₁₉H₂₇Cl₂N₆O₆ (M+H⁺)=m/z 505.1364. Found: m/z 505.1365. HPLC: column: HiQ Sil HS (150×4.60 mm). Mobile phase: isocratic: (0.75 mL/min), 35% MeCN: 65% water. Detection at 280 nm. Retention time: 6.76 minutes, purity: 99.8%.

### N,N′-(4-((2-(2-(2-(2-azidoethoxy)ethoxy)ethoxy)ethyl)carbamoyl)-1,2-phenylene)bis(2-bromoacetamide) (16)

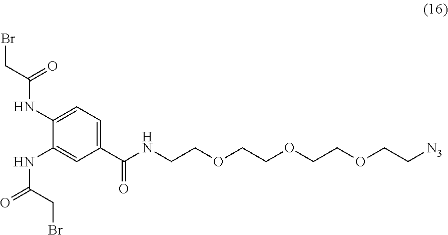 [SMILES: [N-]=[N+]=NCCOCCOCCOCCNC(=O)c1ccc(NC(=O)CBr)c(NC(=O)CBr)c1]

KBr (1.4 g, 12 mmol, 6 equiv.) was added to a solution of N,N′-(4-((2-(2-(2-(2-azidoethoxy)ethoxy)ethoxy)ethyl)carbamoyl)-1,2-phenylene)bis(2-chloroacetamide) (26) (1.0 g, 2.0 mmol) in dry acetone (20 mL). The mixture was refluxed for 4 days. The obtained mixture was filtrated and the solvent was evaporated under reduced pressure. The crude was further purified by silica gel chromatography: (50% to 70% acetone/chloroform) to give the azide-linked acetamide 16 as a white solid (0.5 g, 43%). ¹H NMR (CDCl₃, 500 MHz,): δ 9.02 (s, 1H, NHCH), 8.96 (s, 1H, NHCH), 7.79 (d, J=2.0 Hz, 1H, Ar), 7.70-7.60 (m, 2H, Ar), 7.15 (s, 1H, CONHCH2), 4.09 (d, J=12.2 Hz, 4H, BrCH₂CO), 3.78-3.50 (m, 14H), 3.37 (t, J=5.0 Hz, 2H, CHCH₂N₃). ¹³C NMR (CDCl₃, 126 MHz): δ 166.31, 165.79, 165.13, 133.28, 132.55, 132.28, 125.68, 125.14, 124.79, 70.64, 70.62, 70.52, 70.26, 69.96, 69.63, 50.65, 40.10, 29.71, 29.09, 28.71. ESI-HRMS: Expected for C₁₉H₂₇Br₂N₆O₆ (M+H⁺)=m/z 593.0353. Found: m/z 593.0344. HPLC: column: HiQ Sil HS (150×4.60 mm). Mobile phase: isocratic: (0.75 mL/min), 35% MeCN: 65% water. Detection at 280 nm. Retention time: 7.62 minutes, purity: 99.1%.

Compound 17

### N,N′-(4-((2-(2-(2-(2-azidoethoxy)ethoxy)ethoxy)ethyl)carbamoyl)-1,2-phenylene)bis(2-iodoacetamide)(17)

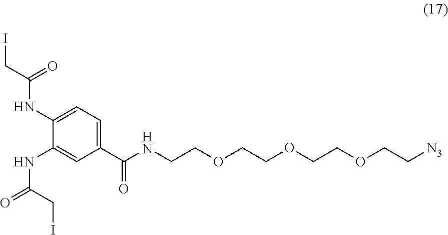 [SMILES: [N-]=[N+]=NCCOCCOCCOCCNC(=O)c1ccc(NC(=O)CI)c(NC(=O)CI)c1]

KI (1.0 g, 8.0 mmol, 4 equiv.) was added to a solution of N,N′-(4-((2-(2-(2-(2-azidoethoxy)ethoxy)ethoxy)ethyl)carbamoyl)-1,2-phenylene)bis(2-chloroacetamide) (26) (1.0 g, 2.0 mmol) in dry acetone (20 mL). The mixture was refluxed for 3 hs. The obtained mixture was filtrated and the solvent was evaporated under reduced pressure. The crude was further purified by silica gel chromatography: (50% to 70% acetone/chloroform) to give the azide-linked acetamide (17) as a yellow solid (1 g, 73%). ¹H NMR (CDCl₃, 500 MHz): δ 9.30 (s, 1 H, 2 x NHCH), 9.17 (s, 1H, NH), 7.61 (s, 1H, Ar), 7.41 (s, 1H, CONHCH2), 7.27 (s, 2H, Ar), 3.93 (d, J=10.8 Hz, 4H, 2 x ICH₂CO), 3.75-3.48 (m, 14H), 3.36 (t, J=5.0 Hz, 2H, CH₂CH₂N₃). ¹³C NMR (CDCl₃, 126 MHz): δ 170.97, 168.13, 166.49, 129.71, 125.31, 125.01, 70.48, 70.31, 70.24, 69.73, 50.60, 40.09, −0.33, −0.53. ESI-HRMS: Expected for C₁₉H₂₇I₂N₈O₆ (M+H⁺)=m/z 689.0076. Found: m/z 689.0108. HPLC: column: HiQ Sil HS (150×4.60 mm). Mobile phase: isocratic: (0.75 mL/min), 35% MeCN: 65% water. Detection at 280 nm. Retention time: 9.10 minutes, purity: 96.2%.

Compound 18

### 3,6,9,12,15,18,21-heptaoxatricosane-1,23-ditosylate (27)

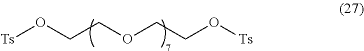

4-Toluenesulphonyl chloride (1.33 g, 7.02 mmol, 2.6 equiv.) was added to a solution of anhydrous pyridine (0.51 g, 6.5 mmol, 2.4 equiv.) and 3,6,9,12,15,18,21-heptaoxatricosane-1,23-diol (1.0 g, 2.7 mmol) in anhydrous DCM (10 mL) and the mixture left to stir under N₂ overnight at room temperature. The solution was then concentrated under reduced pressure and subject to standard work-up (EtOAc). The resultant residue was then purified by silica gel chromatography (40 to 80% EtOAc/petroleum ether) to give 3,6,9,12,15,18,21-heptaoxatricosane-1,23-ditosylate (27) as a colourless oil (1.1 g, 60%). ¹H NMR (CDCl₃, 400 MHz): δ 7.81-7.68 (m, 4H, Ar), 7.34-7.26 (m, 4H, Ar), 4.23-4.01 (m, 4H, 2x SO2OCH₂), 3.70-3.48 (m, 28H), 2.39 (d, J=1.5 Hz, 6H, CHCCH₃). ¹³C NMR (CDCl₃, 101 MHz): δ 144.74, 132.87, 129.77, 127.92, 70.67, 70.54, 70.49, 70.44, 69.20, 68.60, 21.60. ESI-HRMS: Expected for C₃₀H₄₇O₁₃S₂ (M+H⁺)=m/z 679.2453. Found: m/z 679.2448.

### 1,23-diazido-3,6,9,12,15,18,21-heptaoxatricosane (28)

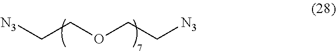

Sodium azide (2 g, 30 mmol, 10 equiv.) was added to a solution of the ditosylate (27) (2.0 g, 3.0 mmol) in DMF and the mixture allowed to stir at 80° C. overnight. The mixture was then concentrated under reduced pressure to remove DMF and the product extracted using ethyl acetate (3×20 mL). The organic extract was then washed with saturated brine solution, dried over MgSO₄, concentrated to give diazide (28) as a colourless oil (1 g, 80.6%). ¹H NMR (CDCl₃, 400 MHz): δ 3.66-3.54 (m, 28H), 3.36-3.28 (m, 4H, 2x N3CH2). ¹³C NMR (CDCl₃, 101 MHz): δ ¹³C NMR δ 70.62, 69.96, 50.60. ESI-HRMS: Expected for C₁₆H₃₃O₇N₆ (M+H⁺)=m/z 421.2405. Found: m/z 421.2466.

### 1,23-diamino-3,6,9,12,15,18,21-heptaoxatricosane (29)

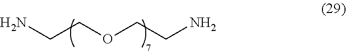

Triphenylphosphine (1.87 g, 7.14 mmol, 3 equiv.) was added portion-wise to a stirred solution of diazide (28) (1.0 g, 2.4 mmol) in dry THF. The reaction was stirred at room temperature overnight. Water (50 mL) was added and the reaction was left stirring at room temperature overnight. The THF was evaporated under reduced pressure and the reaction was filtrated and the filtrate was then washed with DCM (3×100 mL) to remove phosphine oxide. The filtrate was evaporated under reduced pressure to yield diamino-PEG (29) as yellow oil (0.50 g, 57%). ¹H NMR (CDCl₃, 400 MHz,): δ 3.58 (dd, J=2.4, 1.1 Hz, 24H), 3.44 (t, J=5.2 Hz, 4H, 2x NH₂CH₂CH₂O), 2.80 (t, J=5.2 Hz, 4H, 2x NH₂CH₂CH₂O), 1.67 (s, 4H, 2x NH₂). ¹³C NMR (CDCl₃, 101 MHz,): δ 73.27, 70.48, 70.18, 41.68. ESI-HRMS: Expected for C₁₆H₃₇O₇N₂ (M+H⁺)=m/z 369.2600. Found: m/z 369.2850.

### N,N′,N″,N′″-((5,8,11,14,17,20,23-heptaoxa-2,26-diazaheptacosanedioyl)bis(benzene-4,1,2-triyl))tetrakis(2-chloroacetamide) (30)

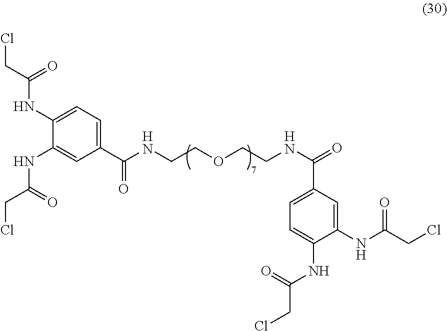

1,23-diamino-3,6,9,12,15,18,21-heptaoxatricosane (29) (150 mg, 0.407 mmol) was added to an anhydrous solution of activated ester (25) (409 mg, 1.02 mmol, 2.5 equiv.) in anhydrous THF. The reaction mixture was then stirred at room temperature for 2 hs before being concentrated under reduced pressure. The crude was purified by silica gel chromatography: (5% to 20% MeOH/DCM) to give the acetamide (30) as a white solid (340 mg, 89%). ¹H NMR (DMSO-d₆, 500 MHz,): δ 9.81 (d, J=25.2 Hz, 4H), 8.54 (t, J=5.6 Hz, 2H), 7.98 (s, 2H), 7.73 (d, J=1.4 Hz, 4H), 4.35 (d, J=9.3 Hz, 8H), 3.64-3.45 (m, 28H), 3.41 (q, J=5.9 Hz, 4H).¹³C NMR (DMSO-d₆, 126 MHz): δ 172.72, 165.38, 165.20, 165.17, 133.26, 131.12, 129.13, 124.98, 124.59, 123.79, 69.71, 69.56, 68.83, 43.28, 43.21. ESI-HRMS: Expected for C₃₈H₅₃Cl₄N₆O₁₃ (M+H⁺)=m/z 941.2419. Found: m/z 941.2451. HPLC: column: HiQ Sil HS (150×4.60 mm). Mobile phase: isocratic: (0.75 mL/min), 35% MeCN: 65% water. Detection at 280 nm. Retention time: 5.83 minutes, purity: 98%.

### N,N′,N″,N′″-((5,8,11,14,17,20,23-heptaoxa-2,26-diazaheptacosanedioyl)bis(benzene-4,1,2-triyl))tetrakis(2-lodoacetamide) (18)

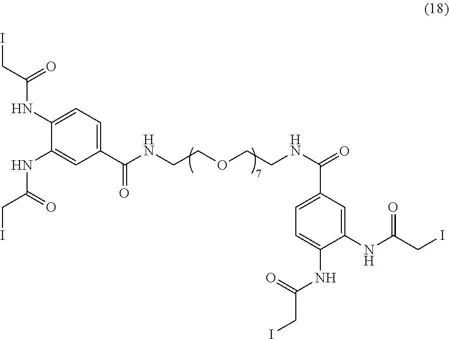

KI (176 mg, 1.06 mmol, 10 equiv.) was added to a solution of N,N′,N″,N′″-((5,8,11,14,17,20,23-heptaoxa-2,26-diazaheptacosanedioyl)bis(benzene-4,1,2-triyl))tetrakis(2-chloroacetamide) (30) (100 mg, 0.106 mmol) in mixture of dry acetone (400 mL). The mixture was refluxed for 3 hs. The resultant mixture was filtrated and the solvent was evaporated under reduced pressure. The crude was purified by silica gel chromatography: (50% to 70% Acetone/Chloroform) to give acetamide (18) as a white solid (130 mg, 93.5%). ¹H NMR (DMSO-d₆, 500 MHz,): δ 9.62 (d, J=16.8 Hz, 4H), 8.31 (d, J=4.9 Hz, 3H), 7.98 (s, 2H), 7.84-7.59 (m, 4H), 3.98 (d, J=7.0 Hz, 8H), 3.75-3.30 (m, 32H). ¹³C NMR (DMSO-d₆, 126 MHz): δ 167.18, 167.09, 165.31, 133.53, 130.78, 129.30, 124.57, 124.29, 123.34, 69.64, 68.87, 1.67, 1.59. ESI-HRMS: Expected for C₃₈H₅₂I₄N₆O₁₃Na₁ (M+Na⁺)=m/z 1330.9669. Found: m/z 1330.9765. HPLC: column: HiQ Sil HS (150×4.60 mm). Mobile phase: isocratic: (0.75 mL/minu), 35% MeCN: 65% water. Detection at 280 nm. Retention time: 9.55 minutes, purity: 98%.

### Example 2

### Aqueous Stability

The stabilities of ester and amide functionalised linker compounds were determined and compared.

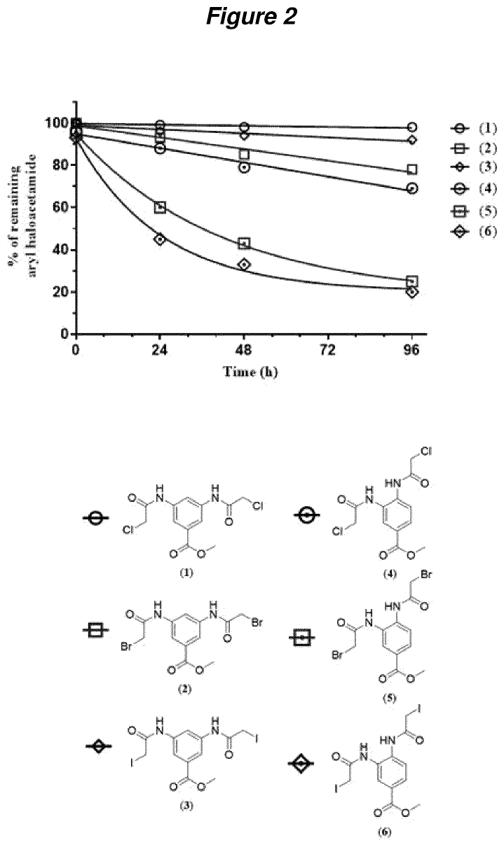

In a first experiment, the aqueous stability of linker compounds 1-6 over 4 days was observed. Stability was determined in phosphate buffer (100 mM, pH 7.5) in the presence of 10% DMF-d7 using ¹H NMR solvent suppression method. The data show that di-haloacetamides that are ortho-substituted (1,2-) have lower aqueous stability than those that are meta-substituted (1,3-). See FIG. 2.

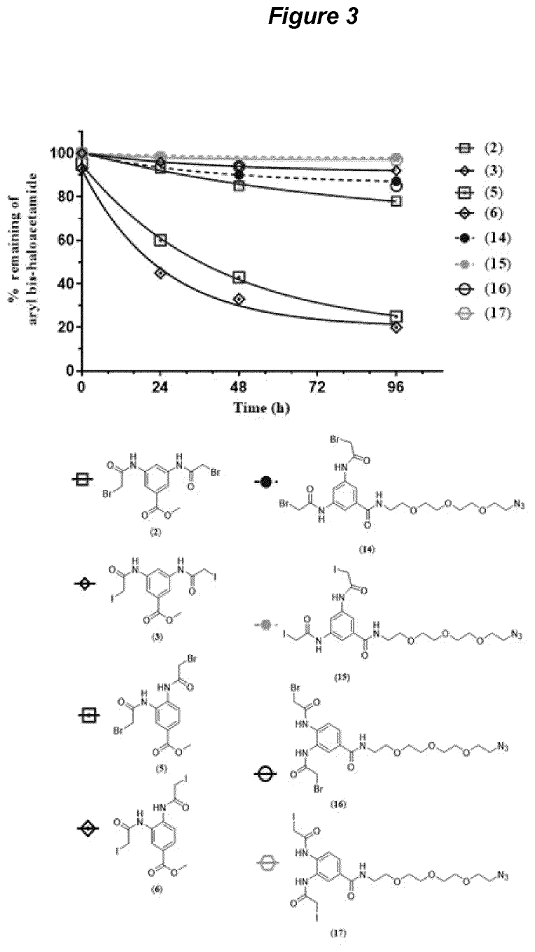

A comparison of the aqueous stability of aryl bis-haloacetamide linkers having ester and amide functionalisation was then made by determining the percentage remaining of the bis-haloacetamide derivatives (2, 3, 5, 6) and 14-16) over 4 days. Stability was again appraised in phosphate buffer (100 mM, pH 7.5) with final concentration of 10% DMF-d7 using solvent suppression method. See FIG. 3.

Surprisingly, the inventors observed that exchanging the ester functional group for an amide functional group reversed the trend for halide stability (iodo- more stable than bromo-) and 3,4-modified were of similar stability to 3,5-modified.

### Example 3

### Reactivity with Amino Acid Thiol Side Chains

The reactivity of the linker compounds of the present invention was tested by reaction with glutathione. The following representative reactions are described. Analogous reactions were performed with amide functionalised linker compounds.

### Reactivity of methyl 3,5-bis(2-haloacetamido)benzoates with glutathione

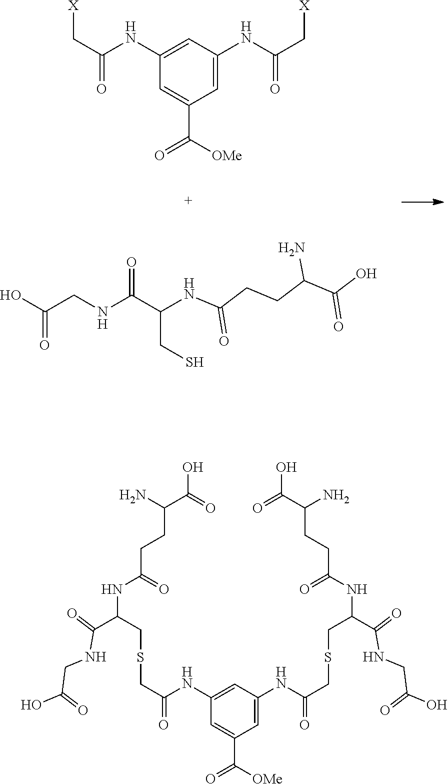

Glutathione (290 mg, 0.943 mmol, 3 equiv.) was dissolved in a solution of aqueous sodium phosphate buffer (100 mM, pH 7.5, 8 mL). Methyl 3,5-bis(2-chloroacetamido)benzoate (1) (100 mg, 0.314 mmol) was dissolved in THF (2 mL), added gradually to glutathione aqueous solution and held overnight at room temperature. The reaction was subsequently concentrated down under reduced pressure and purified by C-18 chromatography (100% H₂O to 20% MeCN/H₂O) to give the product 4.17 as a sticky solid (0.20 mg, 74%). ¹H NMR (D₂O, 500 MHz): δ 7.73 (s, 1H, Ar), 7.56 (s, 2H, Ar), 4.57-4.54 (m, 2H, 2 x NHCHCO), 3.79 (s, 3H, OMe), 3.73-3.63 (m, 6H, 2 x NHCH₂COOH, CH2CHNH₂), 3.40 (s, 4H, 2 x SCH₂CO), 3.13-2.89 (m, 4H, 2 x SCH₂CH), 2.43 (t, J=8 Hz, 4H, 2 x CH₂CH₂CO), 2.06-2.01 (m, 4H, 2 x CH₂CH₂CO). ¹³C NMR (126 MHz, D₂O): δ 176.17, 174.81, 174.00, 171.69, 170.39, 167.65, 137.89, 130.43, 117.32, 54.11, 52.92, 52.89, 43.42, 36.24, 33.77, 31.37, 26.19. HRMS: Expected for C₃₂H₄₄O₁₆S₂N₈Na (M+Na+)=m/z 883.2209 Found: m/z 883.2187. HPLC: column: HiQ Sil HS (150×4.60 mm). Mobile phase: isocratic: (1 mL/min), 10% MeCN: 90% water: 0.1% TFA. Detection at 214 nm. Retention time: 36.39 minutes, purity: 99%.

### Reactivity of methyl 3,4-bis(2-haloacetamido)benzoates with glutathione

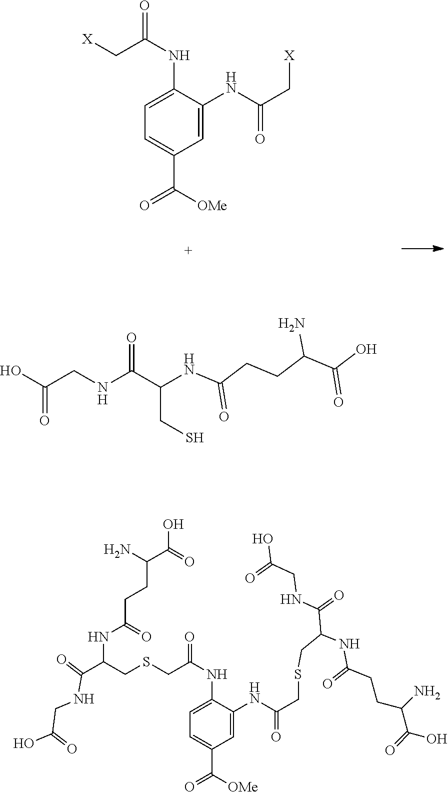

Glutathione (290 mg, 0.943 mmol, 3 equiv.) was dissolved in a solution of aqueous sodium phosphate buffer (100 mM, pH 7.5, 8 mL). Methyl 3,4-bis(2-chloroacetamido)benzoate (4) (100 mg, 0.314 mmol) was dissolved in THF (2 mL), added gradually to glutathione aqueous solution and held overnight at room temperature. The reaction was subsequently concentrated down under reduced pressure and purified by C-18 chromatography (100% H₂O to 20% MeCN/H₂O) to give the product (32) as a sticky solid (0.17 mg, 63%). ¹H NMR (500 MHz, D₂O): δ 8.00 (s, 1H, Ar), 7.93 (dd, J=8.5, 2 Hz, 1H, Ar), 7.63 (d, J=8.5 Hz, 1H, Ar), 4.70-4.56 (m, 2H, 2 x NHCHCO), 3.86 (s, 3H, OMe), 3.76-3.65 (m, 6H, 2 x NHCH₂COOH, CH2CHNH₂), 3.52-3.45 (m, 4H, 2 x SCH₂CO), 3.15-2.90 (m, 4H, 2 x SCH₂CH), 2.47-2.43 (m, 4H, 2 x CH₂CH₂CO), 2.07-2.02 (m, 4H, 2 x CH₂CH₂CO). ¹³C NMR (126 MHz, D₂O): δ 176.11, 174.88, 173.88, 171.63, 171.22, 167.95, 135.56, 128.79, 128.22, 128.04, 125.81, 54.08, 52.90, 52.81, 43.35, 35.72, 35.52, 33.85, 33.79, 31.36, 26.09. HRMS: Expected for C₃₂H₄₄O₁₆S₂N₈Na (M+Na⁺)=m/z 883.2209. Found: m/z 883.2187. HPLC: column: HiQ Sil HS (150×4.60 mm). Mobile phase: isocratic: (1 mL/min), 10% MeCN: 90% water: 0.1% TFA. Detection at 214 nm. Retention time: 14.25 minutes, purity: 99%.

### Reactivity of methyl 2,3-bis(2-bromoacetamido)benzoate with glutathione

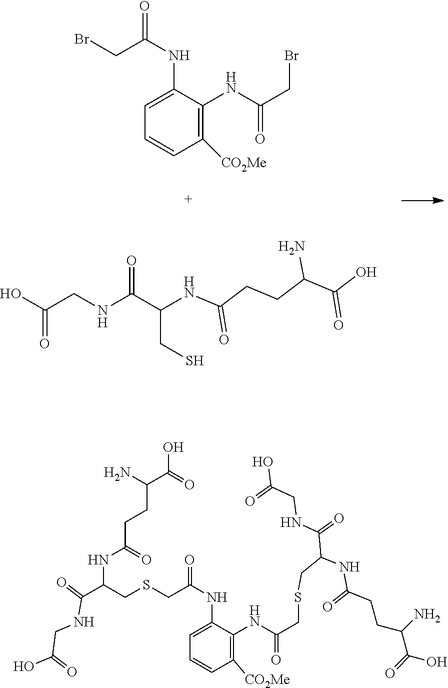 [SMILES: COC(=O)c1cccc(NC(=O)CBr)c1NC(=O)CBr ; "+" ; NC(CCC(=O)NC(CS)C(=O)NCC(=O)O)C(=O)O ; COC(=O)c1cccc(NC(=O)CSCC(NC(=O)CCC(N)C(=O)O)C(=O)NCC(=O)O)c1NC(=O)CSCC(NC(=O)CCC(N)C(=O)O)C(=O)NCC(=O)O]

Glutathione (0.11g, 3.66mmol, 3eq.) was dissolved into 6mL of phosphate buffer 0.1 M pH=7 and added to 4 ml of Methyl 2,3-bis(2-bromoacetamido)benzoate (7) (0.05 g, 1.22 mmol, 1 eq.) solution in THF. The mixture was kept under stirring at RT for 24 hours. Reverse phase column (C18) was used to purify the obtained product and the fractions obtained with 10% Acetonitrile/water afforded the product (0.05 g, 48%).

Comparison of Amide Functionalised Compounds

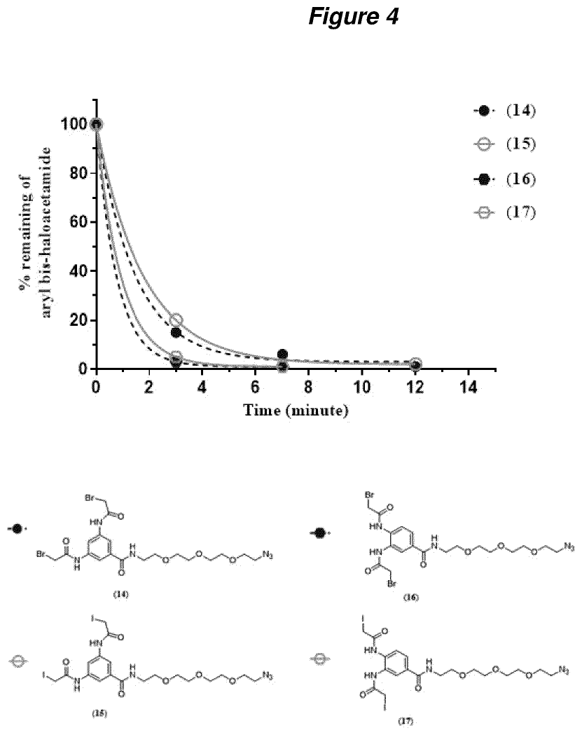

The inventors compared the reactivity of various linker compounds of the invention by determining the percentage remaining of the bis-haloacetamide derivatives 14-17 in the presence of glutathione (2.2 equiv.) in aqueous phosphate buffer (100 mM, pH 7.5). See FIG. 4.

They observed that, despite no difference between 3,4- and 3,5-amide modified derivatives for hydrolysis (reaction with water), there is a significant difference between their reaction with the thiol of glutathione. The 3,4-modified derivatives react faster than 3,5-modified compounds with thiols.

The ester compounds reacted so quickly a reaction rate could not be determined.

### Example 4

### Reaction of Bromo Acetamide Linkers with Trastuzumab

Linker compounds 2, 5, 9 and 12 were reacted with Trastuzumab under varying stoichiometries and the reaction products analysed using SDS-PAGE.

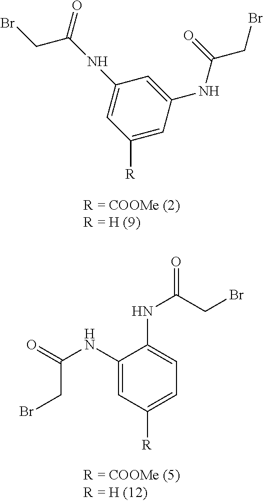

The reactions used fully reduced Tmab (0.034 mM) in Tris.HCI buffer (100 mM, 0.15 mM NaCl, 5 mM EDTA, pH 7.5) with bis-bromoacetamide linkers. Tmab (0.033 mM) was reduced with 5 equiv. of TCEP for 2 hs at room temperature.

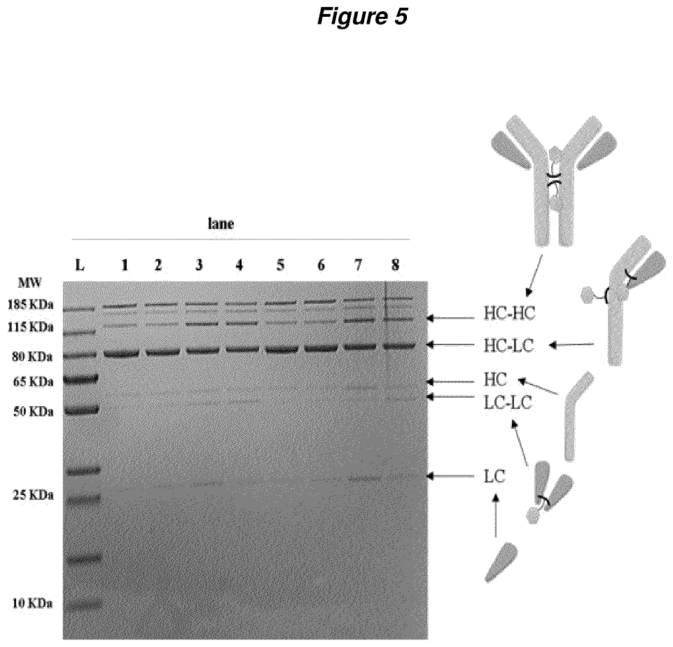

The analysis and attributed products are shown in FIG. 5.

L: protein ladder,

lane 1: Tmab incubated with 5 equiv. of methyl 3,4-bis(2-bromoacetamido)benzoate (5),

lane 2: Tmab incubated with 8 equiv. of methyl 3,4-bis(2-bromoacetamido)benzoate (5),

lane 3: Tmab incubated with 5 equiv. of methyl 3,5-bis(2-bromoacetamido)benzoate (2),

lane 4: Tmab incubated with 8 equiv. of methyl 3,5-bis(2-bromoacetamido)benzoate (2),

lane 5: Tmab incubated with 5 equiv. of N,N′-(1,2-phenylene)bis(2-bromoacetamide) (12),

lane 6: Tmab incubated with 8 equiv. of N,N′-(1,2-phenylene)bis(2-bromoacetamide) (12),

lane 7: Tmab incubated with 5 equiv. of N,N′-(1,3-phenylene)bis(2-bromoacetamide) (9),

lane 8: Tmab incubated with 8 equiv. of N,N′-(1,3-phenylene)bis(2-bromoacetamide) (9).

Key: HC: heavy chain, LC: light chain, LC-LC: light chain homodimers, HC-HC: heavy chain homodimers. Protein samples were resolved by reducing SDS-PAGE (4-12% gel).

The inventors observed a different pattern of thiol bridging between the ortho-(1 ,2- and 3,4-) and meta-(1,3- and 3,5-) substituted linking compounds. When excess (5 or 8 equivalents) of linker is used, the meta-substituted linkers are less selective as more LC-LC (47 kDa) and HC-HC (106 kDa) are produced. The major product for all reactions is the HALF-ANTIBODY conjugate, HC-LC (75 kDa) with 2 linkers attached.

The product of reaction with linker compound 5 (labelled HC-LC) was analysed by mass spectrometry. Cross-linked Tmab with methyl 3,4-bis(2-bromoacetamido) benzoate (5) showed a major peak at 74,528.03 Da, and expansion of the spectrum at the 74 KDa region showed major peaks at 74,528.03 Da and 74,689.24 Da (glycoforms). This mass spectrometric analysis of the crude reaction product confirms the attachment of two linkers, one linker bridging intra-HC-HC cysteine residues of the hinge region.

FIG. 1(1) shows a schematic representation of the half-antibody produced with two rebridged disulfide bonds, intra-chain heavy-heavy and heavy-light chains (labelled HC-LC). FIG. 1(5) is chemically identical to the structure shown in FIG. 1(1), but represents the physical state in which the half-antibody may be observed. In size-exclusion chromatography, the product will appear to have a molecular weight of 150 kDa, but each covalent bonded structure has a weight of 75 kDa (half-antibody) as represented by FIG. 1(1).

Analysis of the reaction product of reaction with unfunctionalised linker compound 12 confirmed attachment of two linkers, with one linker again bridging intra-HC-HC cysteine residues of the hinge region.

Deconvoluted spectrum protein MS of cross-linked Tmab with N,N′-(1,2-phenylene)bis(2-bromoacetamide) (12). The MS spectrum of cross-linked Tmab showed a major peak at 74,572.57 Da, while expansion of the spectrum at 74 KDa region showed major peaks at 74,572.57 Da and 74,410.91 Da.

### Example 5

### Influence of pH on selectivity of bis-(2-bromoacetamide)-linkers

The influence of pH on reaction with bis-(2-bromoacetamide) linking compounds was investigated by conducting an SDS-PAGE analysis of cross-linking of fully reduced Tmab (0.033 mM) in Tris.HCI buffer (100 mM, 0.15 mM NaCl, 5 mM EDTA, pH 6, 7.5, or 8) incubated with bis-haloacetamide linkers. Tmab (0.033 mM) was reduced with 5 equiv. of TCEP for 2 h at room temperature.

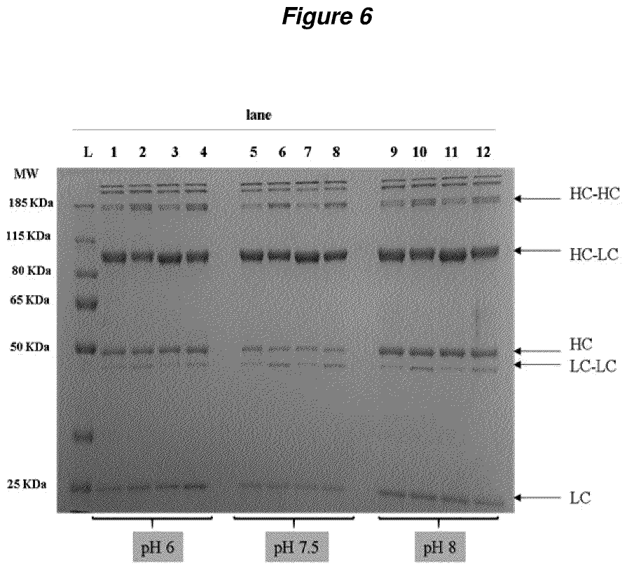

The analysis and attributed products are shown in FIG. 6.

L: protein ladder,

Lane 1, 5 and 9: Tmab incubated with 5 equiv. of methyl 3,4-bis(2-bromoacetamido)benzoate (5),

Lane 2, 6 and 10: Tmab incubated with 5 equiv. of methyl 3,5-bis(2-bromoacetamido)benzoate (2),

Lane 3, 7 and 11: Tmab incubated with 5 equiv. of N,N′-(1,2-phenylene)bis(2-bromoacetamide) (12),

Lane 4, 8 and 12: Tmab incubated with 5 equiv. of N,N′-(1,3-phenylene)bis(2-bromoacetamide) (9).

HC: heavy chain, LC: light chain, LC-LC: light chain homodimers, HC-HC: heavy chain homodimers. Protein samples were resolved by reducing SDS-PAGE (10% gel).

The best levels of conjugation were achieved at pH=7.5. In all cases, ortho-(1,2- and 3,4-) substituted linker compounds are more selective (less LC-LC and inter-HC-HC) than the meta-(1,3- and 3,5-) modified linker compounds.

### Example 6

### Reaction of excess bis-(2-iodoacetamide) linkers with Trastuzumab

Linker compounds 3, 6, 10 and 13 were reacted in excess with Trastuzumab and the reaction products analysed using SDS-PAGE.

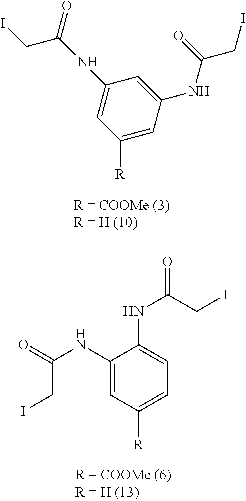

The reactions used fully reduced Tmab (0.033 mM) in Tris.HCI buffer (100 mM, 0.15 mM NaCl, 5 mM EDTA, pH 7.5) with bis-iodoacetamide linkers. Tmab (0.033 mM) was reduced with 5 equiv. of TCEP for 2 hs at room temperature.

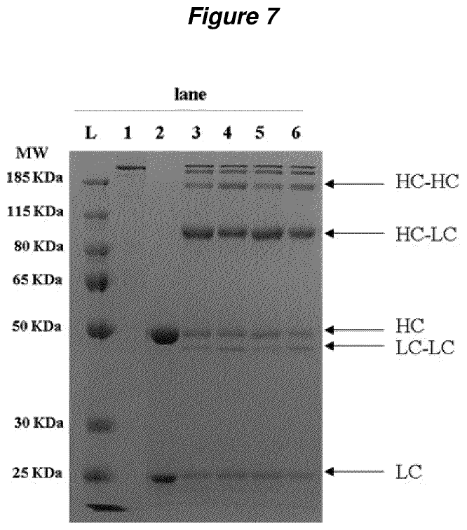

The analysis and attributed products are shown in FIG. 7.

L: protein ladder,

lane 1: Tmab control (Non-reducing dye),

lane 2: Tmab control (reducing dye),

lane 3: Tmab incubated with 5 equiv. of methyl 3,4-bis(2-iodooacetamido)benzoate (6),

Lane 4: Tmab incubated with 5 equiv. of methyl 3,5-bis(2-iodoacetamido)benzoate (3),

Lane 5: Tmab incubated with 5 equiv. of N,N′-(1,2-phenylene)bis(2-iodoacetamide) (13),

Lane 6: Tmab incubated with 5 equiv. of N,N′-(1,3-phenylene)bis(2-bromoacetamide) (10).

HC: heavy chain, LC: light chain, LC-LC: light chain homodimers, HC-HC: heavy chain homodimers. Protein samples were resolved by reducing SDS-PAGE (10% gel).

HC: heavy chain, LC: light chain, LC-LC: light chain homodimers, HC-HC: heavy chain homodimers. Protein samples were resolved by reducing SDS-PAGE (10% gel).

Similar selectively was observed as for the analogous bromo linker compounds.

### Example 7

### Selectivity of Reaction of Amide Substituted Linkers with Trastuzumab

The reaction of amide functionalised linker compounds 14, 15, 16 and 17 with Trastuzumab was investigated.

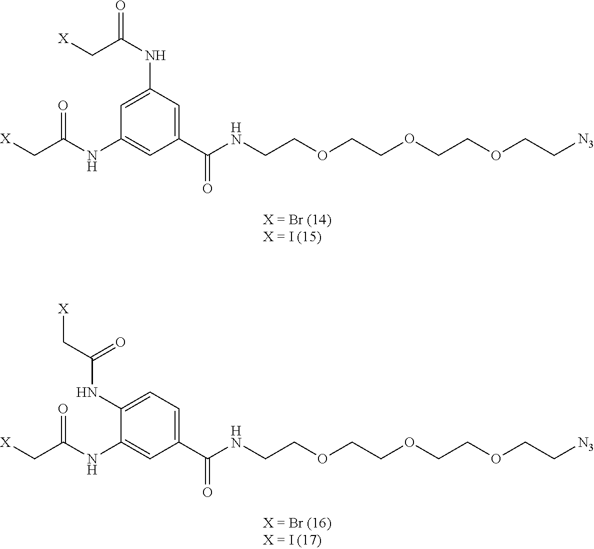

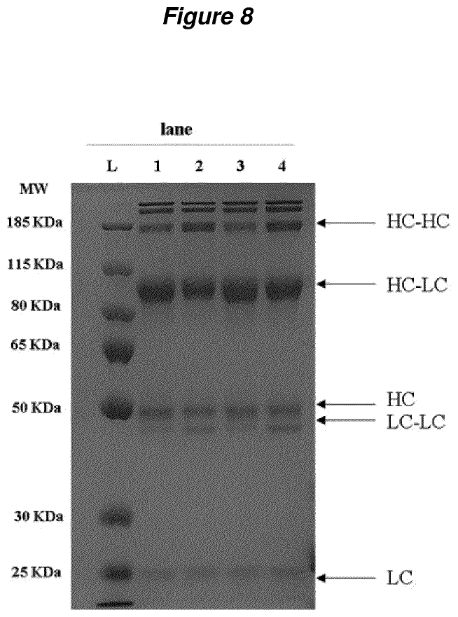

SDS-PAGE analysis of cross-linking of reduced Tmab (0.013 mM) in Tris.HCl buffer (0.5 M, pH 7.5, 5 mM EDTA) with 5 equiv. of TCEP at room temperature overnight is shown in FIG. 8.

L: ladder,

lane 1: Tmab incubated with 5 equiv. of 16,

lane 2: Tmab incubated with 5 equiv. of 14,

lane 3: Tmab incubated with 5 equiv. of 17,

lane 4: Tmab incubated with 5 equiv. of 15.

Deconvoluted spectrum protein MS of rebridged Tmab with Compound 17 linker showing a major peak at 75,059.82 Da confirmed the attachment of two linkers to the half-antibody product.

### Example 8

### Selectivity with Other Monoclonal Antibodies

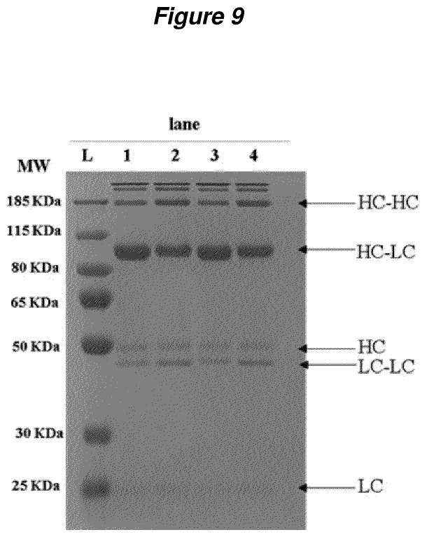

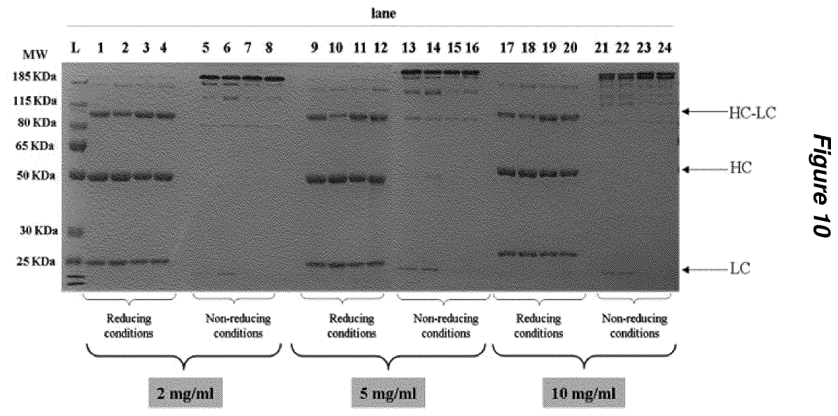

SDS-PAGE analysis of cross-linking of fully reduced Rituximab (0.035 mM) in Tris.HCl buffer with bis-bromoacetamide linkers is shown in FIG. 9. Rituximab (0.035 mM) was reduced with 5 equiv. of TCEP for 2 hrs at room temperature.

L: protein ladder,

lane 1: Rmab incubated with 5 equiv. of methyl 3,4-bis(2-bromoacetamido)benzoate (6),

lane 2: Rmab incubated with 5 equiv. of methyl 3,5-bis(2-bromoacetamido)benzoate (3),

lane 3: Rmab incubated with 5 equiv. of N,N′-(1,2-phenylene)bis(2-bromoacetamide) (12),

lane 4: Rmab incubated with 5 equiv. of N,N′-(1,3-phenylene)bis(2-bromoacetamide) (9).

HC: heavy chain, LC: light chain, LC-LC: light chain homodimers, HC-HC: heavy chain homodimers. Protein samples were resolved by reducing SDS-PAGE (10% gel).

Similar selectivity and reaction products observed to reaction with Trastuzumab. 1,2-compounds show less LC-LC and HC-HC products with more effective formation of HC-LC product at 75kDa. Results observed using Trastuzumab can therefore be considered a reasonable exemplar for antibodies.

### Example 9

### Selective Functionalisation of Antibodies

The inventors have demonstrated that selectivity of linkers can be exploited to selectively mono functionalise and hetero-bi-functionalise antibodies such as Trastuzumab.

The site selective functionalisation of Trastuzumab was investigated. FIG. 10 shows SDS-PAGE analysis of cross-linking of partially reduced Tmab in Tris.HCl buffer (100 mM, 0.15 mM NaCl, 5 mM EDTA, pH 6, 7.5, or 8) with bis-bromoacetamide linkers. Tmab was reduced with 1.1 equiv. of TCEP for 2 hs at 4° C. and incubated with 1.1 equiv. each linker at room temperature overnight.

L: protein ladder,

Lanes 1, 9 and 17: 1 equiv. of methyl 3,4-bis(2-bromoacetamido)benzoate (5),

Lanes 2, 10 and 18: 1.1 equiv. of methyl 3,5-bis(2-bromoacetamido)benzoate (2),

Lanes 3, 11 and 19: 1.1 equiv. of N,N′-(1,2-phenylene)bis(2-bromoacetamide) (12),

Lanes 4, 12 and 20: 1.1 equiv. of N,N′-(1,3-phenylene)bis(2-bromoacetamide) (9).

Lane 5-8, 13-16, and 21-24 represent the same reactions as above, in the same order described, but run under non-reducing conditions (non-reducing dye).

HC: heavy chain, LC: light chain, LC-LC: light chain homodimers, HC-HC: heavy chain homodimers. Protein samples were resolved by reducing SDS-PAGE (10% gel).

Equivalent intensity of bands at 75kDa and 50kDa indicate selective functionalisation of a single disulphide bridge to produce one HC-LC conjugate per mAb. In other words, monofunctionalisation. Unfunctionalized linkers 12 and 9 appear to perform better, as fewer lower molecular weight bands are seen under non-reducing conditions, indicating reaction has proceeded to a greater extent.

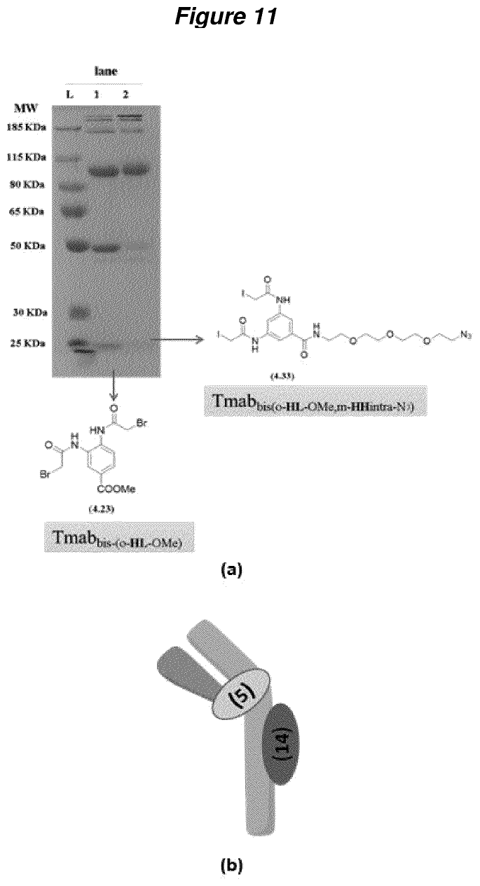

Through sequential partial reduction, incubation, further reduction and incubation the selective hetero-bi-functionalisation of Trastuzumab was effected. FIG. 11a shows SDS-PAGE of bifunctional cross-linking of Tmab (0.033 mM) in Tris.HCl buffer (100 mM, 0.15 mM NaCl, 5 mM EDTA, pH 7.5) using sequential method. Tmab (0.033 mM) was reduced with 2.2 equiv. of TCEP for 2 hs and incubated with 2.2 equiv. of methyl 3,4-bis(2-bromoacetamido)benzoate (5) at room temperature overnight (lane 1). Functionalised Tmab (0.033 mM) was then further reduced with 2.2 equiv. of TCEP for 2 hs and incubated with 4 equiv. of N,N′-(5-((2-(2-(2-(2-azidoethoxy)ethoxy)ethoxy)ethyl)carbamoyl)-1,3-phenylene)bis(2-iodoacetamide) (15) at room temperature overnight (lane 2).

Protein samples were resolved by reducing SDS-Page (10% gel).

The deconvoluted spectrum protein MS shows a major peak at 74,711.99 Da corresponding to reaction with one molecule of linker 5 and one molecule of linker 15.

This is a demonstration of the ability to form hetero-bi-functionalised half-antibodies through sequential addition of 2 different linkers.

The structure is stylistically represented in FIG. 11b. Linker 5 shows rebridging of the disulphide that links the light and heavy chains of the antibody. Linker 15 is referred to herein as ‘intra-chain’ rebridging, linking the two thiols that made up the hinge region of the antibody.

### Example 10

### Formation of Stable Thio-Bridged Fab Derivatives and Selective Preparation of a mAb-Protein Conjugate

The linker compounds of the invention are able to form stable thio-bridged Fab derivatives. These can be subsequently conjugated to reduced or partially reduced antibodies or other proteins. First, IFab was buffer exchanged to conjugation Tris.HCl buffer (pH 7.5) and diluted to (5 mg/mL, 0.1 μmol) using Amicon® Ultra-0.5 mL (3 KDa) centrifugal filters. Stock solution of TCEP (4.0 mg/mL, 0.042 mmol, 14 mM) was prepared in the same conjugation buffer. IFab was reduced by incubation with TCEP (2 equiv. relative to IFab) for 1 h at room temperature. Then a TCEP quenching step using penta-PEG azide for 1 h was followed.

The reduced IFab was incubated with bis-o-diiodoacetamide (PEG)₇ linker 18 (10 μL of stock solution (7 mg/mL), 5 equiv.) for 3 hs at room temperature.

Excess reagent was removed by using quick step of purification using protein A column (1 mL HisTrap, FF) to remove excess linkers from functionalized IFab solution prior to conjugation with partially reduced Tmab. The binding buffer Tris.HCI (20 mM, 500 mM NaCl, pH 7.4) was used to collect Fab fractions, then the collected faction were concentrated using were concentrated using Amicon® Ultra-15 mL (3 KDa) centrifugal filters and finally buffer exchanged into conjugation buffer.

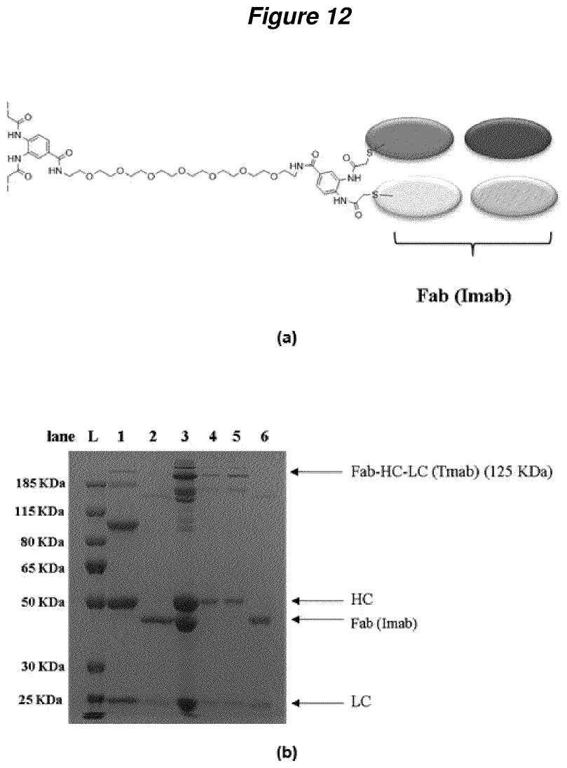

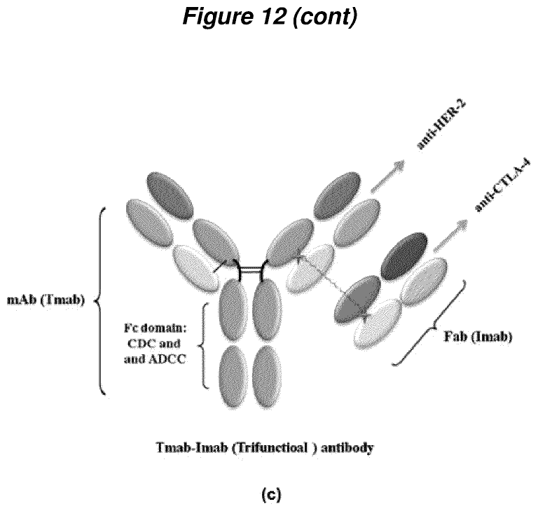

A deconvoluted protein MS spectrum of IfAb control (non-reduced) showed major peaks at 47,636.32 Da, while the deconvoluted protein MS spectrum of functionalised IfAb with bis-o-diiodoacetamide (PEG)7 linker (18) showed major peaks at 48,690.90 Da, confirming the functionalised had occurred. As schematic structure is shown in FIG. 12a.

Tmab was buffer exchanged to Tris.HCl (100 mM, 150 mM NaCl, 5 mM EDTA, pH 7.5) and diluted to (5.0 mg/mL, 0.03 μmol) using Amicon® Ultra-0.5 mL (10 KDa) centrifugal filters. Tmab was reduced by incubation with TCEP (1.1 equiv. relative to Tmab) for 2 hs at 40. Functionalised IFab (4 equiv. relative to Tmab) was then added to the reduced Tmab and left at room temperature overnight. SEC was then performed using superdex column (HiLoad 16/600, Superdex 200 pg, GE Healthcare) for purification of reaction products. Prior to loading onto the column, samples were centrifuged at 20,000 g for 10 minutes. The collected fractions containing the conjugate were buffer exchanged into conjugation buffer and sterilised using 0.45 μm membrane filters.

See FIG. 12b.

L: protein ladder

Lane 1: Tmab incubated with 1.1 equiv. of methyl 3,4-bis(2-bromoacetamido)benzoate (5) as control,

Lane 2: IfAb conjugate,

Lane 3: Tmab conjugation with IfAb at room temperature overnight showing the correct estimated mass of the conjugate around 125 KDa with significant reduction of the 75 KDa band (half-antibody) before performing size exclusion chromatography,

Lane 4: size exclusion column purification collected fractions (F6-G2),

Lane 5: size exclusion column purification collected fractions (G4-G13),

Lane 6: size exclusion column purification collected fractions (H1-H7).

Lanes 4 and 5 contain the desired mAb-fAb conjugate (some HC-HC impurity in Lane 5). A schematic representation of the antibody conjugate is shown in FIG. 12c. It may also be termed a mAb-protein conjugate or tri-functional monoclonal antibody. The inventors have demonstrated its selective preparation and stability to AKTA purification on ProteinA column.

### Example 11

### Selectivity of Thio-Bridging Compounds is Dependent on Their Regio-Chemistry: Ortho Substituted Versus Meta Substituted

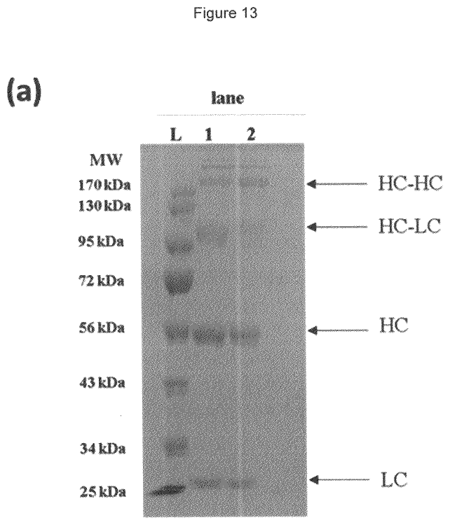

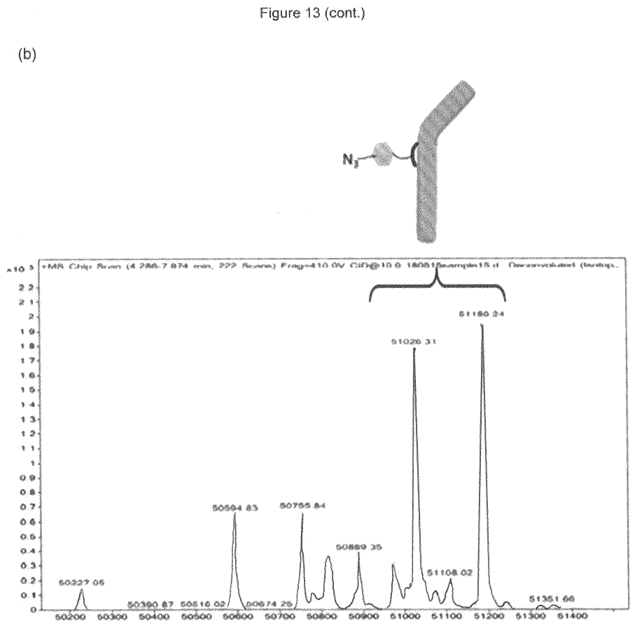

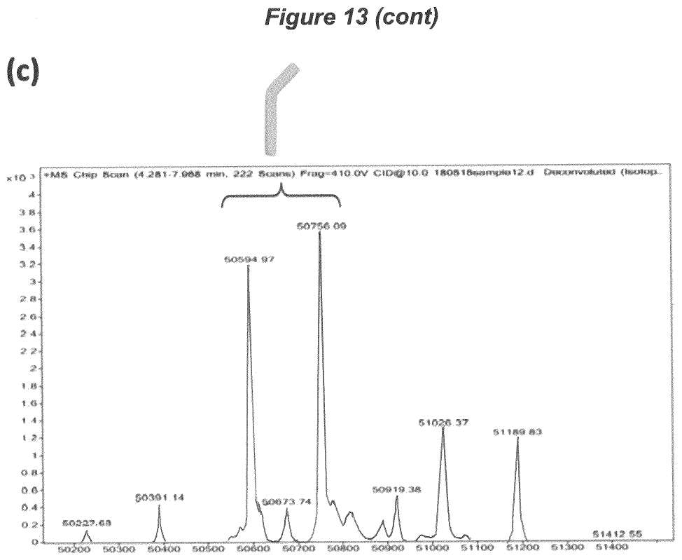

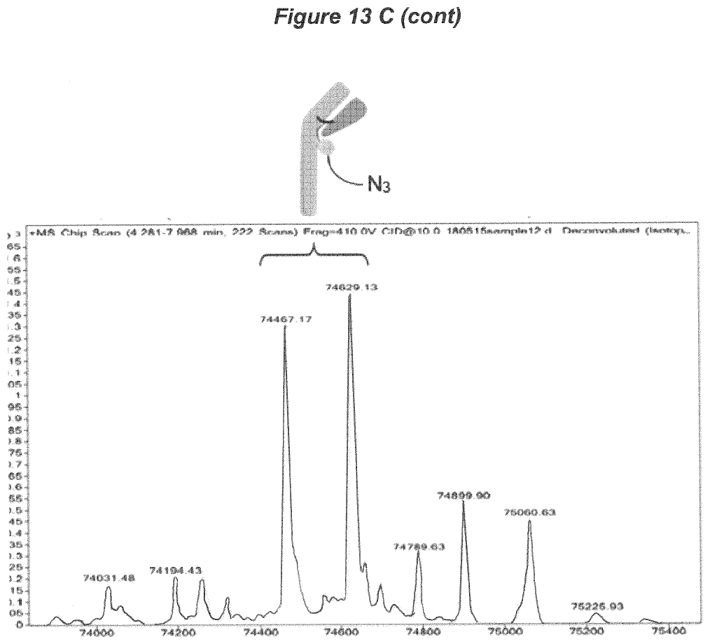

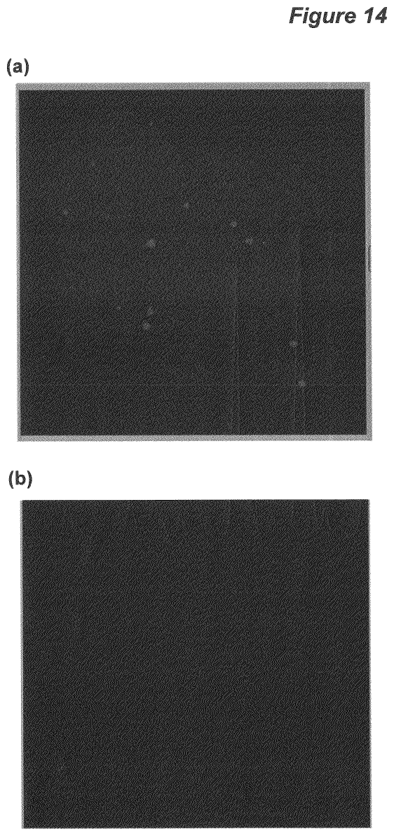

Selectivity of compounds 15 and 17 was evaluated where fully reduced Tmab (4 equiv.) was incubated with only 2 equivalents of each bis-iodoacetamide linker 15 and 17. The reaction products were resolved using SDS-PAGE analysis (shown in FIG. 13a) and further characterised by protein MS (shown in FIG. 13b and c).

First, Tmab was buffer exchanged into Tris.HCl buffer (pH 7.5) and diluted to (5 mg/mL, 34 μM, 1 ml) using Amicon® Ultra-0.5 mL (10 kDa) centrifugal filters. Tmab was reduced by incubation with TCEP (4 equiv. relative to Tmab) for 2 h at 4° C. The reduced protein was aliquoted into 100 μL samples for each reaction. A stock solution of N,N-(4-((2-(2-(2-(2-azidoethoxy)ethoxy)ethoxy)ethyl)carbamoyl)-1,2-phenylene)bis(2-iodoacetamide) (17) was prepared in DMF at final concentration (1.0 mg/mL, 1.4 μmol, 1.4 mM). Working solution of 17 (0.68 mg/mL) was prepared by serial dilution with DMF. 2 equiv. of working solution of 17 (7 μL, 2 equiv. relative to Tmab aliquot) was added to the reduced protein and held at room temperature overnight. An analogous procedure was followed for the reaction of compound 15 with Tmab.

The azide modified bis-iodoacetamide compounds 15 and 17 displayed an interesting manner of selectivity. SDS-PAGE analysis of the reaction with the meta-substituted compound 15 showed the HC (half-antibody bridged intra) of Tmab being the major protein product present (FIG. 13a, lane 2). Characterisation with protein MS confirmed that this HC protein product has undergone conjugation with one molecule of 15 through rebridging of the intra-chain heavy-heavy disulphide (FIG. 13b).

In contrast, the ortho-substituted compound 17 displayed greater preference to cross-link heavy-light disulfide bonds with a significant amount of a higher molecular weight product observed by SDS-PAGE analysis (FIG. 13a, Lane 1). Characterisation with protein MS confirmed that this protein product has undergone HC-LC disulphide rebridging with one molecule of 17 (FIG. 13c). Importantly, MS analysis also demonstrated that the remaining HC protein was native, and had not undergone any appreciable conjugation with compound 17.

FIG. 13a shows an SDS-PAGE gel (10%) evaluating selectivity of bis-iodoacetamide linkers in cross-linking Tmab (5 mg/mL, 34 μM) in Tris.HCI buffer (100 mM, pH 7.5) containing 150 mM NaCl, and 5 mM EDTA, reduced with 4 equiv. of TCEP (136 μM) and incubated with (6.8 μM, 2 equiv.) of each bis-iodooacetamide linker at room temperature overnight.

L: protein ladder,

Lane 1: Tmab incubated with 2 equiv. of N,N′-(4-((2-(2-(2-(2-azidoethoxy)ethoxy)ethoxy)-ethyl)carbamoyl)-1,2-phenylene)bis(2-iodoacetamide) (17),

Lane 2: Tmab incubated with 2 equiv. of N,N′-(4-((2-(2-(2-(2-azidoethoxy)ethoxy)ethoxy)ethyl)-carbamoyl)-1,3-phenylene)bis(2-iodoacetamide) (15).

Deconvoluted protein MS spectra of Tmab cross-linked with 2 equiv. of N,N-(4-((2-(2-(2-(2-azidoethoxy)ethoxy)ethoxy)ethyl)carbamoyl)-1,3-phenylene)bis(2-iodoacetamide) (15) (FIG. 13b) and N,N′-(4-((2-(2-(2-(2-azidoethoxy)ethoxy)ethoxy)ethyl)carbamoyl)-1,2-phenylene)bis(2-iodoacetamide) (17) (FIG. 13c).

### REFERENCES

A number of publications are cited above in order to more fully describe and disclose the invention and the state of the art to which the invention pertains. Full citations for these references are provided below. The entirety of each of these references is incorporated herein.

EP333573

S M Andrew and J A Titus Current Protocols in Cell Biology (2000) 16.4.1-16.4.10.

E J Smith, L Visai, S W Kerrigan, P Speziale, and T J Foster* Infect Immun. 2011, 79(9): 3801-3809.

T Kantner, B Alkhawaja, A G Watts (2017) ACS Omega, 2, 5785-5791.

For discussion of protecting groups and their synthesis and use, see Peter G. M. Wuts and Theodora W. Greene, Greene's Protective Groups in Organic Synthesis 4^{th} Ed 2006, Wiley-Blackwell.

For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press.

1. A linker compound of Formula (I) or a salt thereof: 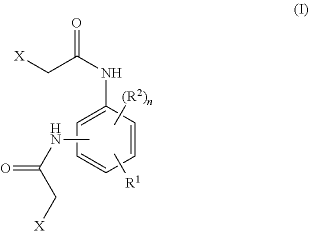
wherein
each X is independently F, Cl, Br, or I;
R¹ is H, COOR^{A}, CONH₂, CONHR^{A}, CONR^{A}₂, CONHL, or CONR^{A}L;
L, if present, is a chain terminating in a reactive group R³;
each R^{A}, if present, is independently selected from C₁₋₄alkyl;
n is 0, 1, 2, or 3; and
each R², if present, is independently selected from F, Cl, Me, CF₃, OMe, and OCF₃ or R² is a group as defined for R¹.

2. The compound of claim 1 wherein L is a polyether or polythioether terminating in R³ and R³ is selected from N₃, a group comprising a C≡C bond, a protected amine, a leaving group and a moiety of Formula (H): 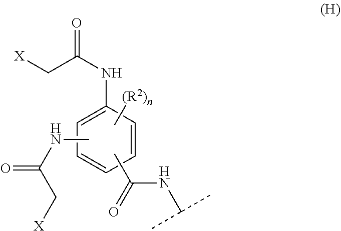
wherein
each X is independently F, Cl, Br, or I;
n is 0, 1, 2, or 3; and
each R², if present, is independently selected from F, Cl, Me, CF₃, OMe, and OCF₃ or R² is a group as defined for R¹ in claim 1.

3. The compound of claim 1 wherein n is 0.

4. The compound of claim 1, wherein X is Br or I.

5. The compound of claim 1, wherein the compound is of Formula (IIa), (IIb) or (IIc): 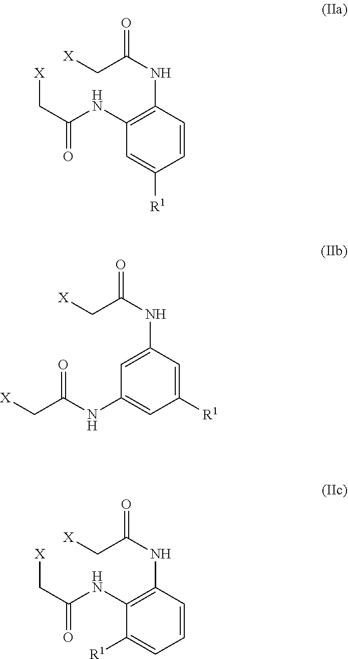
wherein
X is Br or I; and
R¹ is H, COOMe, CONH₂, or CONHL.

6. The compound of claim 1, wherein the compound is of Formula (IVa), (IVb) or (IVc) 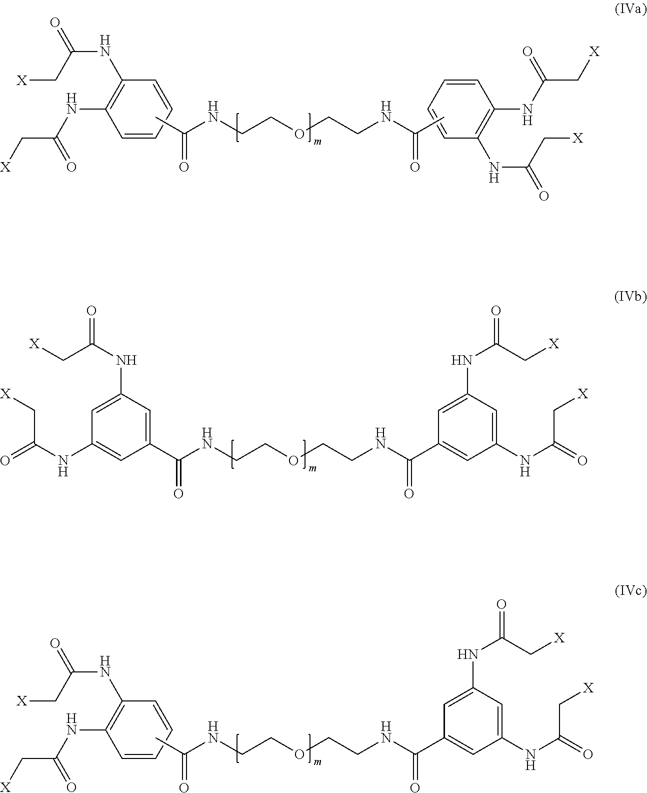
optionally wherein:
each X is Br; or
each X is I; or
two X one arylene are Br and two X or the other arylene are I.

7. The compound of claim 1, wherein the compound is selected from Examples (1) to (18):

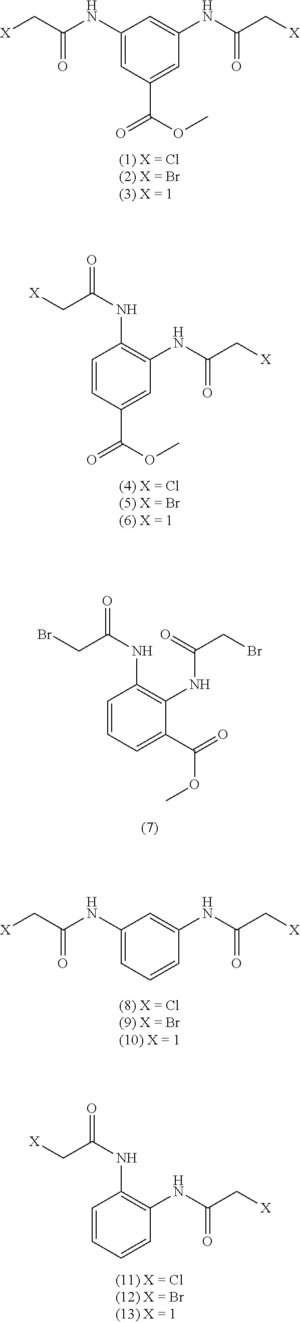

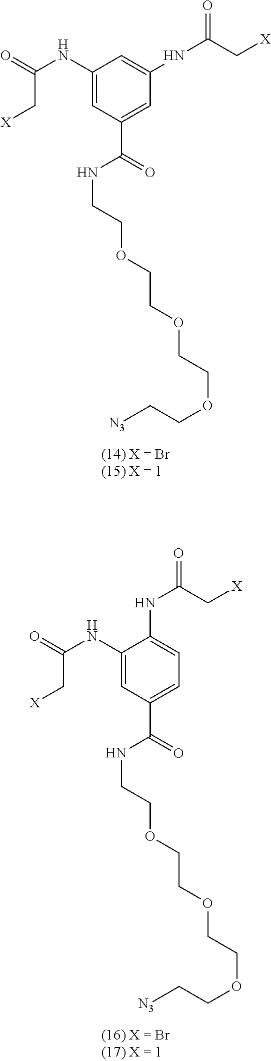

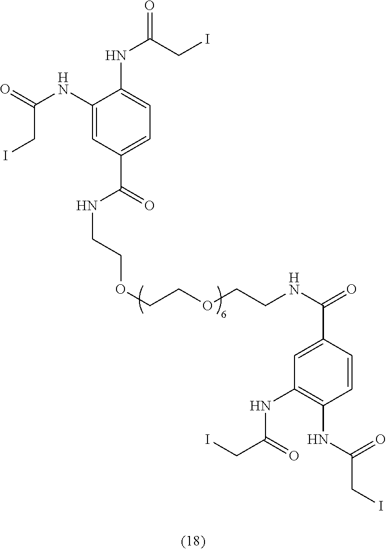

8. A half-antibody comprising a linker residue bridging intra- the heavy chain-heavy chain cysteine residues of the hinge region, wherein the linker residue is a moiety as defined in claim 1 with two X displaced by thiol groups.

9. A mono-functionalized antibody comprising a linker residue bridging the heavy and light chains, wherein the linker residue is a moiety as defined in claim 7, with two X displaced by thiol groups.

10. A thio bridged fab antibody fragment comprising a linker residue which is a moiety as defined in claim 1 with two X displaced by thiol groups.

11. The thio bridged fab antibody fragment of claim 10, wherein the linker residue is a moiety of Formula (IXa), (IXb), (IXc) or (IXd): 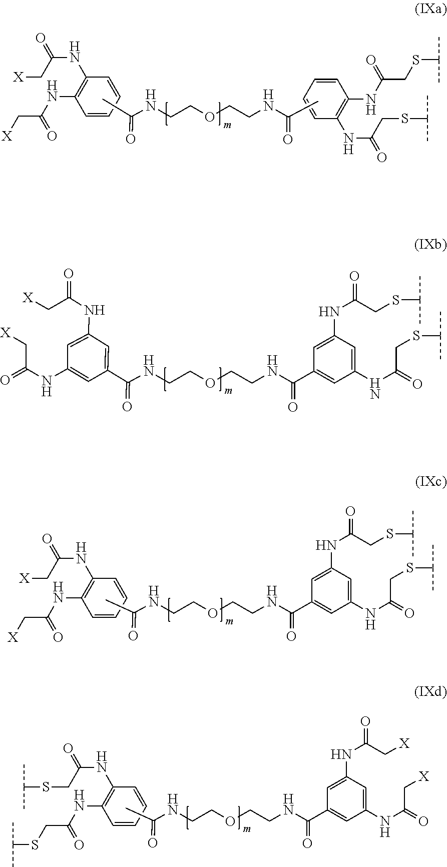
wherein X is Br or I; and where m is selected from 3, 4, 5, 6, 7, 8, 9, and 10 and [symbol] represents a point of attachment to the protein chain.

12. A method for producing a mono-functionalized antibody comprising a linker residue bridging the heavy and light chains, wherein the linker residue is a moiety of Formula (Ia) with two X displaced by thiol groups, the method comprising treating a fully reduced antibody with about 1 to 1.1 equivalents of a linker compound of Formula Ia: 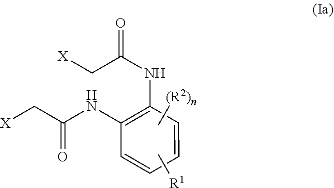
wherein X is Br; preferably wherein n is 0.

13. A method for producing a mono-functionalized antibody comprising a linker residue bridging the heavy and light chains, wherein the linker residue is a moiety as defined in claim 1 with two X displaced by thiol groups, the method comprising:
(i) providing a partially reduced antibody by reducing an antibody using about 1 to 1.1 equivalent of a reducing agent; then
(ii) treating said partially reduced antibody with said linker compound.

14. . A method of producing a half-antibody comprising a linker residue bridging intra- the heavy chain-heavy chain cysteine residues of the hinge region and a linker residue bridging the light and heavy chains, wherein the linker residue is a moiety as defined in claim 1 with two X displaced by thiol groups, the method comprising treating a fully reduced antibody with at least 4 equivalents of said linker compound.

15. A method of producing a half-antibody wherein the linker residue is a moiety of Formula (Ib) bridging intra-HC-HC cysteine residues of the hinge region, the method comprising treating a fully reduced antibody with about 2 to 2.2 equivalents of a linker compound of Formula (Ib): 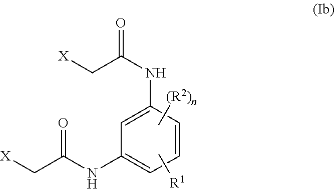
wherein X is I; preferably wherein n is 0 and/or preferably wherein the R¹ group and two haloacetamide groups are arranged in a 1,3,5 configuration; optionally wherein the method comprises, after treatment of the fully reduced antibody with about 2 to 2.2 equivalents of a linker compound of Formula (Ib), treatment with a further linker compound according to the invention.

16. A method of producing a hetero-bi-functionalised half-antibody having a first linker residue bridging intra- the heavy chain-heavy chain cysteine residues of the hinge region and a second linker residue bridging the light and heavy chains, wherein each linker residue is a moiety as defined in claim 1 with two X displaced by thiol groups, the method comprising:
(i) treating a partially reduced antibody with said first linker compound to produce a first conjugate; then
(ii) further reducing said first conjugate to produce a reduced conjugate; then
(iii) treating said reduced conjugate with said second linker compound, wherein the first and second linker compounds are different, to produce said hetero-bi-functionalised half-antibody conjugate.

17. The method of claim 12, wherein at least one of the linker compounds includes L and the method includes a step of attaching a further moiety selected from an antibody, half-antibody, antibody fragment, protein, polypeptide, drug or a fluorescent or radio label.

18. A method of producing a Fab-Mab or Fab-protein conjugate, the method comprising treating a reduced or partially reduced antibody or a protein with a thio bridged fab antibody fragment according to claim 10.

19. The method of claim 12, wherein R¹ is H, COOMe, CONH₂, or CONHL and n is 0.

20. The half-antibody of claim 8, wherein R¹ is H, COOMe, CONH₂, or CONHL and n is 0.
